(19) United States
(12) Patent Application Publication
(10) Pub. No.: US 2026/0209710 A1
(43) Pub. Date: Jul. 23, 2026

(54) Species-Specific Tissue Culture Medium for the Propagation of Cells

(71) Applicant: SymbioCellTech, LLC, Salt Lake City, UT (US)

(72) Inventors: Christof Westenfelder, Salt Lake City, UT (US); Anna Maddock Gooch, Salt Lake City, UT (US)

(21) Appl. No.: 18/997,284

(22) PCT Filed: Jul. 19, 2023
(86) PCT No.: PCT/US2023/070472

§ 371 Date: Jan. 21, 2025

Related U.S. Application Data
(60) Provisional application No. 63/390,842, filed on Jul. 20, 2022.

(51) Int. Cl. C12N 5/0775; A61K 35/28; A61K 35/39; C12N 5/071

(52) U.S. Cl. CPC C12N 5/0667; A61K 35/28; A61K 35/39; C12N 5/0663; C12N 5/0676; C12N 2500/32; C12N 2500/38; C12N 2500/60; C12N 2501/11; C12N 2501/115; C12N 2501/135; C12N 2501/33; C12N 2501/39; C12N 2501/392

(57) ABSTRACT

A cell culture medium is disclosed. The cell culture medium comprises: a species-specific cell culture medium comprising: bFGF of the species, PDGF-BB of the species, and EGF of the species; and serum of the species. Further disclosed is a canine cell culture medium comprising: canine bFGF, canine PDGF-BB, and canine EGF; and canine serum. A composition is described comprising: a medium comprising: basal medium, sodium bicarbonate, HEPES, L-glutamine, L-alanine, Insulin, hydrocortisone, progesterone, Asc-2-p, holo-transferrin; bFGF of the species, PDGF-BB of the species, and EGF of the species; and serum consistent with the cells of the species; MSC cells of the species. A method of producing exosomes is described, the method comprising: expanding mesenchymal stem cells and/or islet cells in culture, the culture and collecting exosomes released by the mesenchymal and/or adipose stem cells in culture.

### CROSS REFERENCE TO RELATED APPLICATIONS

This application is a national phase entry under 35 U.S.C. § 371 of International Patent Application PCT/US2023/070472, filed Jul. 19, 2023, designating the United States of America and published in English as International Patent Publication WO2024/020431 on Jan. 25, 2024, which claims the benefit under Article 8 of the Patent Cooperation Treaty to U.S. Provisional Patent Application Ser. No. 63/390,842, filed Jul. 20, 2022, the entireties of which are hereby incorporated by reference.

### TECHNICAL FIELD

The present invention relates to species specific culture medium for culture and propagation of mesenchymal stromal cells, adipose derived mesenchymal stromal cells, pancreatic islet cells and other cells, and isolation of exosomes from various cells.

### BACKGROUND

Cell culture media provide the environment to grow, propagate, and maintain cells in a controlled, artificial and in vitro environment. Human beings and many other species are increasingly the recipients of cellular therapeutics, particularly Mesenchymal Stromal Cells (MSCs), for the treatments of various disorders, including renal diseases, bone and joint disorders, diabetes, and immune diseases. These therapies require isolation, purification and often culture of MSCs and/or other cells over many doublings. Such culture requires the use of serum supplemented culture medium, and given the susceptibility humans and other animals, particularly dogs to foreign protein reactions, ideally, the serum-supplement is derived from the donor and recipient species so that cells that are introduced into the recipient are not carriers of xenogeneic proteins that might cause an antigenic response in the recipient. Currently, standard culture media are typically supplemented with serum concentrations of 10-20% (v/v).

The culture medium formulations described here are xeno-free, and require significantly less serum than standard culture media, and are designed and tailored for the culture of cells from multiple species (human, dog, cat, horse, etc.). In order to prevent xenogeneic immune responses, this medium contains species-specific or identical at the amino-acid level proteins, and species-specific serum, but no xenogeneic animal proteins. Further, the reduction in the need for serum lowers both the ethical burden and the monetary cost of culturing cells.

### SUMMARY

In a first aspect, the disclosure provides a species-specific cell culture medium comprising: basal medium, sodium bicarbonate, HEPES, L-glutamine, L-alanine, hydrocortisone, progesterone, Asc-2-p, holo-transferrin; bFGF of the species, PDGF-BB of the species, insulin of the species, and EGF of the species; and serum of the species.

In a second aspect, the disclosure provides a canine cell culture medium comprising: basal medium, sodium bicarbonate, HEPES, L-glutamine, L-alanine, insulin, hydrocortisone, progesterone, Asc-2-p, holo-transferrin; canine bFGF, canine PDGF-BB, and canine EGF; and canine serum.

In a third aspect the disclosure provides a composition comprising: a medium comprising: basal medium, sodium bicarbonate, HEPES, L-glutamine, L-alanine, insulin, hydrocortisone, progesterone, Asc-2-p, holo-transferrin; bFGF of the species, PDGF-BB of the species, and EGF of the species; and serum consistent with the cells of the species; mesenchymal stromal cells of the species.

In a fourth aspect the disclosure provides a composition comprising: a medium comprising basal medium, sodium bicarbonate, HEPES, L-glutamine, L-alanine, insulin, hydrocortisone, progesterone, Asc-2-p, holo-transferrin; bFGF of the species, PDGF-BB of the species, and EGF of the species; and serum consistent with the cells of the species; islet cells of the species.

In a fifth aspect the disclosure provides a method of preparing a species-specific cell culture medium comprising: providing basal medium, sodium bicarbonate, HEPES, L-glutamine, L-alanine, insulin, hydrocortisone, progesterone, Asc-2-p, holo-transferrin; providing bFGF of the species, PDGF-BB of the species, and EGF of the species; and providing serum of the species.

In a sixth aspect the disclosure provides a method of preparing a cell culture medium comprising: providing basal medium, sodium bicarbonate, HEPES, L-glutamine, L-alanine, insulin, hydrocortisone, progesterone, Asc-2-p, holo-transferrin; providing canine bFGF, canine PDGF-BB, and canine EGF; and providing canine Serum.

In a seventh aspect the disclosure provides a method for culturing and propagating cells: providing a cell culture medium comprising: basal medium, sodium bicarbonate, HEPES, L-glutamine, L-alanine, insulin, hydrocortisone, progesterone, Asc-2-p, holo-transferrin; species specific bFGF, species specific PDGF-BB, and species specific EGF; and species-specific Serum; providing cells for propagation; providing conditions for propagating cells.

In an eighth aspect the disclosure provides a method of producing exosomes, the method comprising: expanding cells in culture, the culture medium comprising: providing basal medium, sodium bicarbonate, HEPES, L-glutamine, L-alanine, insulin, hydrocortisone, progesterone, Asc-2-p, holo-transferrin; providing species specific bFGF, species specific PDGF-BB, and species specific EGF; and providing species specific serum; and collecting exosomes released by the cells in culture.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a view of Cell Morphologies of Dog 1 ASCs comparing their morphologies when cultured in standard culture medium (left) to that of the species-specific medium disclosed herein (right).

FIG. 2 is a view of Cell Morphologies of Dog 2 ASCs comparing their morphologies when cultured in standard culture medium (left) to that of the species-specific medium disclosed herein (right).

FIG. 3 is a view of Cell Morphologies of Dog 3 ASCs comparing their morphologies when cultured in standard culture medium (left) to that of the species-specific medium disclosed herein (right).

FIG. 4 is a view of Cell Morphologies of Dog 4 ASCs comparing their morphologies when cultured in standard culture medium (left) to that of the species-specific medium disclosed herein (right).

FIG. 5 is a view of Cell Morphologies of Dog 5 ASCs comparing their morphologies when cultured in standard culture medium (left) to that of the species-specific medium disclosed herein (right).

FIG. 6 is a view of Cell Morphologies of Dog 6 ASCs comparing their morphologies when cultured in standard culture medium (left) to that of the species-specific medium disclosed herein (right).

FIG. 7 is a graph of population doublings (PDLs) for dog MSCs from 6 different dogs cultured 4 days in either standard culture medium containing 10% (v/v) dog serum or SCT's dog culture medium. The figure demonstrates the more consistent and higher cell yields obtained with SCT's medium vs. standard medium, despite significantly lower serum concentration in SCT's medium. *, Differences between cells cultured in traditional, serum containing medium and SCT's medium are statistically significant (P=0.01; two-tailed, paired T-test)

FIG. 8 is a graph of Doubling Times for dog ASCs from 6 different dogs cultured 4 days in either standard culture medium containing 10% (v/v) dog serum or SCT's dog culture medium. The figure demonstrates the more consistent and faster growth rates obtained with SCT's medium vs. standard medium, despite significantly lower serum concentration in SCT's medium. *, Differences between cells cultured in traditional, serum containing medium and SCT's medium are statistically significant (P<0.05; two-tailed, paired T-test)

FIG. 9 is a graph of doubling times of cells from the 6 donors in Table 1 cultured in SCT255 vs. standard medium over 5-16 doublings. Doubling times are faster and less varied for ASCs cultured in SCT255 than for those same ASCs cultured in standard, 10% (v/v) serum containing culture medium.

FIGS. 10A-10B shows that Cytokine expression profiles of dog ASCs cultured in SCT's medium are similar to those of ASCs cultured in standard culture medium. In FIG. 10A, cytokine profiles of each dog are shown separately. In FIG. 10B the mean cytokine profile of N=6 dogs is shown. In both FIG. 10A and FIG. 10B, gene expression was normalized to that of cells cultured in DMEM+10% (v/v) dog serum (standard medium). IGF-1 and IL-6 expression trend higher in cells cultured in SCT's medium than in normal medium, but do not reach statistically significant levels (log 10[RQ]≥±2). IGF-1 expression by ASCs has pro-regenerative and immune modulatory actions, and IL-6 is also immune modulatory (1,2).

FIGS. 11A-11B demonstrates that dog ASCs cultured in SCT255c maintain gene up-regulation of IDO-1 and other anti-inflammatory and immune modulatory cytokines upon exposure to INF-γ, just as cells cultured in standard medium+10% (v/v) dog serum, but that expression of the immune-modulatory gene PD-L2 is consistently and significantly up-regulated when dog ASCs are cultured in SCT255c. In FIG. 11A, dog ASCs from 6 different donors were cultured in SCT255c+INF-γ [10 mcg/ml] overnight. Gene expression was normalized to control samples of the same cells cultured in the same medium but without INF-γ. Differences of log 10(RQ)>±2 are considered statistically significant. Exposure of dog ASCs that are cultured in SCT255c to INF-γ results in normal up-regulation of immune-modulatory cytokine genes. In FIG. 11B, Dog ASCs from 6 different donors were cultured in SCT255c+INF-γ overnight. Expression was normalized to control samples of the same cells cultured in standard, 10% (v/v) dog serum containing medium+INF-γ. Except for PD-L2, differences are not statistically significant [log 10(RQ)>±2]. This figure demonstrates that culture of dog ASCs in SCT255c provides a superior immune-modulatory cytokine profile of the cells than does culture in standard medium+10% (v/v) dog serum.

FIG. 12 is a graph showing PD-L2 gene expression of dog ASCs from 6 different donors cultured in either SCT255c or standard culture medium+10% (v/v) dog serum. PD-L2 was expressed in only 1 dog cell line cultured in standard culture medium but in all 6 tested lines cultured in SCT255c. Shown are Cycle Thresholds normalized to internal controls (housekeeping genes) for ASCs from 6 dogs. These data demonstrate that culture of dog ASCs in SCT255c results in a superior immune-modulatory cytokine profile of the cells than does culture in standard medium+10% (v/v) dog serum.

FIG. 13 is a graph showing PD-L1 protein expression on dog ASCs from 6 different donors cultured in either SCT255c+INF-γ or standard culture medium+10% (v/v) dog serum+INF-γ. PD-L1 protein expression was assessed by FACS and graphed as the percentage of cells expressing the protein. These data, again, demonstrate that culture of dog ASCs in SCT255c results in a superior immune-modulatory cytokine profile of the cells than does culture in standard medium+10% (v/v) dog serum.

FIGS. 14A-14D are graphs depicting Growth rates, cell yields of Islet Cells (ICs) from N=6 dogs cultured in SCT255c vs. standard culture medium and demonstrate the faster growth rates and superior yields of another cell type—dog islet cells—when cultured in SCT255c vs standard culture medium+20% (v/v) dog serum.

FIGS. 15A-15B are graphs depicting Islet endocrine gene expression in P0 and P1 dog ICs (N=6) cultured in SCT255c vs. standard culture medium, indicating that islet cell identity is preserved when the cells are cultured in SCT255c.

FIG. 16A is a micrograph and illustration that normal capillaries—ones where there is no underlying microvascular disease state—can accommodate relatively large Mesenchymal Stem Cells (~100 μm diameter), FIG. 16B is a micrograph of Mesenchymal Stem Cell-derived Exosomes (~40-100 nm diameter), and an illustration that capillaries with microvascular disease (relatively occluded compared to normal capillaries) can accommodate the relatively smaller exosomes, which contain the MSC cargo, better than they can accommodate MSCs.

FIGS. 17A-17C: Protein concentration, nanoparticles per million, and nanoparticle mean and mode sizes for extracellular vesicles collected from dog ASCs cultured in SCT255c base medium vs standard base medium. ASCs from 4 different dogs were cultured in each of 2 separate experiments. Differences in protein concentration (FIG. 18A), a reflection of extracellular vesicle, or particle, number (FIG. 18B), were significantly different between the two control medium experiments, but not for the SCT255c medium experiments (FIG. 18A). Mean sizes of particles collected from cells cultured in SCT255c did not differ significantly from those cultured in standard medium (FIG. 18C). The mode size did not differ from the mean size for cells cultured in SCT255c indicating the extracellular vesicles collected were more consistent and uniform. However the mode size of particles (extracellular vesicles) collected from standard medium differed significantly from the mean, indicating a substantial variation of extracellular vesicles (FIG. 18C).

FIG. 18: a graph showing IFG-1 mRNA cargo in extracellular vesicles collected from dog ASCs cultured in SCT255c is significantly upregulated compared with that of extracellular vesicles collected from dog ASCs cultured in standard medium.

FIGS. 19A-19B are micrographs showing the growth of Human MSCs in SCT255 h medium compared to a control medium.

FIGS. 20A-20B are micrographs showing the growth of Human 041508 cells in SCT255 h medium compared to a control medium.

FIGS. 21A-21B are micrographs showing the growth of Human “Rooster” cells in SCT255 h medium compared to a control medium.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein “MSC” is meant to refer to Mesenchymal Stromal Cells. Which are pluripotent cells found in nearly all tissues, and can differentiate into many different types of cells, such as chondrocytes, osteoblasts, adipocytes, and potentially others. MSCs also interact with immune cells and can modulate immune responses including enabling immunosuppression and tolerance induction. They have excellent proliferation, differentiation, and immunoregulatory capacities. Mesenchymal stem cells can self-renew by dividing and can differentiate into multiple tissues including bone, cartilage, muscle, and fat cells, and connective tissue.

As used herein, “ASC” is meant to refer to Adipose Stromal Cells. ASCs are one type of mesenchymal stromal cells, adipose-derived mesenchymal stromal cells. ASC can easily be harvested from adipose tissue in large quantities. Adipose Stromal Cells taken from subcutaneous adipose tissue are often referred to as ASCs. ASCs are post-embryonic, self-renewing cells, capable of giving rise to a variety of parenchymal cells in vitro when adequately stimulated. ASCs readily form colonies and form stromal progeny in vitro, when transplanted they can express general fibroblastic markers, and modulate the host immune system. ASCs are in most instances considered to have limited life span in recipients, as they are transient cells. Potential benefits include an affinity for sites of damage and inflammation, where the secretion of trophic factors influence the repair of damaged tissues. As used herein, M/ASC refers to MSCs and/or ASCs. As used herein, “diabetes” is meant to refer to diseases that result in too much sugar in the blood.

As used herein, “Type 1 diabetes” is meant to refer to a chronic condition in which the pancreas produces little to no insulin.

As used herein, “Type 2 diabetes” is meant to refer to a chronic condition affecting the way the body processes blood sugar.

As used herein, “Treating” or “treatment” means that the symptoms of the underlying disease are at least reduced, and/or that one or more of the underlying cellular, physiological, or biochemical causes or mechanisms causing the symptoms are reduced and/or eliminated. It is understood that reduced, as used in this context, means relative to the state of the disease, including the molecular state of the disease, not just the physiological state of the disease. Treating or treatment does not require a complete cure.

As used herein “therapeutically effective amount” is an amount sufficient to act as a treatment as defined above. This may be determined by, e.g., standard techniques used to monitor and/or diagnose a particular disease state.

As used herein “extracellular vesicles” is meant to refer to types of extracellular vesicles that contain particles (proteins, DNA, and RNA) of the cells that secrete them. Extracellular vesicles fuse with other cells where the particles are taken up and can affect cell function and behavior. Examples of extracellular vesicles include, but are not limited to, exosomes, mitosomes, and oncosomes.

In describing the composition and manufacture of a reduced serum, xeno free cell culture medium certain components are described with reference to the company of manufacture and the specific product used. These specific products are for illustration of the components within the cell culture medium and are not limiting descriptions as to the use of similar components.

The present invention provides reduced serum, xeno-free cell culture medium for use in mammalian cell culture for the production of species-specific M/ASCs, islet cells, and other cell types.

Treatments for diseases and disorders continue to advance. Among the advances for disease treatment are cellular therapies. Cellular therapies are in most instances understood to be the transplantation or infusion of cells into a patient to repair or replace damaged or diseased tissues. Many types of cells are used in these therapies including M/ASC's and islet cells. While cellular therapies can use cells taken from a donor subject and be directly transplanted or infused into a recipient subject, the number of cells needed for effective results is often greater than the number of cells harvested from the donor. Additionally, to provide cost-effective treatment it is in most instances more beneficial to culture and propagate cells for use in these therapies than to extract them from a donor subject and transplant them into a recipient subject for each transplant, infusion, or treatment.

A large number of common, acute and chronic diseases are characterized, at both early and advanced stages, by the development of similar microvascular pathologies that lead to progressive organ injury, organ loss, morbidity, and mortality. The intrinsic capillary/microvascular network of all organs and tissues is critical to their physiological function and overall health. Capillaries facilitate blood supply, oxygenation and removal of metabolites from all organs. Anatomically, they are substantially composed of endothelial cells, smooth muscle cells, and pericytes. The latter “monitor” and maintain the function and anatomical integrity of all capillaries.

When pathological processes such as diabetes mellitus, inflammatory, auto-immune, degenerative, age-related, trauma and other injuries affect the function of the capillary complex, blood supply, tissue oxygenation and removal of metabolites from affected organs is impaired, translating into permanent loss of function and systemic morbidity and mortality.

Mesenchymal Stem or Stromal Cells (MSC) from bone marrow, fat, umbilical cord and other sources are non-embryonic, “adult” stem cells that possess potent anti-inflammatory, anti-apoptotic, immune modulating, angiogenic and vasculo-protective, anti-fibrotic and anti-thrombotic paracrine activities that have been used for the promising treatment of various acute and chronic diseases and organ injuries. MSCs in culture release beneficial cytokines and growth factors that mediate their pleiotropic effects.

It is possible to isolate MSCs from every postnatal tissue, however, fat tissue and bone marrow are considered prime sources for MSCs because of the ease of isolation in these tissues. MSCs isolated from fat or Adipose tissue are referred to as ASCs. As adult tissues have ceased differentiating the numbers of M/ASCs in adult tissues are low. Growing M/ASCs in culture assists in producing sufficient quantities of M/ASCs for therapeutic uses. The heterogeneity of M/ASCs is affected by isolation methods and culture conditions; including culture reagents, culture vessels, and culture environment. Growth media for both isolation and expansion of M/ASCs from fat tissue and bone marrow have been described. There are many commercially available base culture media, which, in order to be used as cell culture media, typically require pH adjustment and supplementation with mammalian serum at a concentration of 10 to 20% (v/v). FBS is one of the most common serum supplements for the media. FBS provides growth factors, attachment factors, and various other nutrients. The concentrations, qualities, and sources or species of origin of these factors and nutrients vary between suppliers and even between batches from the same supplier. The aforementioned variations in nutrients and factors adds to the heterogeneity of M/ASCs and other cells' numbers and quality. Especially within regulatory frameworks this heterogeneity is undesirable. Additionally, the introduction of proteins that are xenogeneic to the cells being cultured is problematic for several reasons. First, it is our hypothesis that cells will propagate best, most consistently and most reproducibly when using proteins derived from their species of origin. Such proteins will fit their intended targets as they have evolved to, which proteins from another species may or may not do, depending upon how conserved in nature such proteins are. Second, proteins present in culture medium are often taken up by the cells being cultured and stored in those cells. Issues have be seen with human MSCs cultured with FBS as the serum source, and then used therapeutically. While the MSCs themselves are typically immune privileged, in a case where FBS was used to culture the cells for therapeutic use, bovine protein was taken up by and stored in the cells, and subsequently an immune response to the bovine proteins was detected in individuals treated with the cells (3).

The invention provides a cell culture medium, which includes, in whole or in part, a modified basal medium. Modified basal cell media may be derived from standard basal cell media known in the art. Suitable basal media include but are not limited to Minimal Essential Medium (MEM) Dulbecco's Modified Eagle Medium (DMEM), DMEM/F12, Basal Medium Eagle (BSE), or any of a variety of other media commercially available. In addition to the basal medium the culture medium includes Sodium Bicarbonate, HEPES (4-(2-hydroxyethyl)-1-piperazineethanesulfonic acid), GlutaMax™ (L-glutamine, L-alanine), lipid concentrate (such as Gibco chemically defined lipid concentrate), insulin, hydrocortisone (e.g. plant, from wild yam), progesterone (e.g. synthetic), putrescine (e.g. microbial), ASC-2-P (Vitamin C), bFGF (corresponding to the species of the cells), PDGF-BB (corresponding to the species of the cells), EGF (corresponding to the species of the cells), insulin, and serum (corresponding to the species of the cells). In certain embodiments, the insulin may be of the same species as the cells cultured.

Cells need nutrients and a conducive environment to proliferate. The necessary nutrients include both organic nutrients, such as lipids, carbohydrates, amino acids, and vitamins; inorganic nutrients, such as salts and minerals; and non-nutrient factors, such as hormones and growth factors.

There are several chemically defined lipid concentrates available on the market. These lipid concentrates are concentrated lipid emulsions designed to reduce or replace fetal bovine serum in cell culture media for a wide variety of applications. One example of lipid concentrate is Gibco chemically defined lipid concentrate, which can be purchased from ThermoFisher Scientific (Catalog No. 11905031). This lipid concentrate contains the following components; arachidonic acid, 2.0 mg/L; cholesterol, 220.0 mg/L; DL-alpha-tocopherol acetate, 70.0 mg/L; ethyl alcohol; linoleic acid, 10.0 mg/L; linolenic acid, 10.0 mg/L; myristic acid, 10.0 mg/L; oleic acid, 10 mg/L; palmitic acid, 10.0 mg/L; palmitoleic acid, 10.0 mg/L; pluronic F-68, 90000.0 mg/L (a non-ionic detergent for protecting cells from hydrodynamic damage); stearic acid, 10.0 mg/L; Tween 80®, 2200.0 mg/L (a non-ionic detergent used in selective protein extraction and isolation of nuclei from mammalian cell lines).

Glutamine is an essential amino acid for protein and nucleic acid synthesis and for energy production, therefore including it in the medium increases the function of many cell types. However, glutamine, is difficult to include because is extremely unstable at physiological pH and non-enzymatically degrades to ammonia. GlutaMax™ (L-glutamine, L-alanine); Invitrogen, Carlsbad, CA), a stabilized form of L-glutamine, is a substitute for glutamine and is more stable in aqueous solutions.

Growth factors need to be included in the media. Those growth factors include but are not necessarily limited to Fibroblast Growth Factor basic (bFGF) of the species of the cells being cultured, Platelet Derived Growth Factor (PDGF) of the species of the cells being cultured, Transforming Growth Factor-β1 (TGF-β1) of the species of the cells being cultured, and Epidermal Growth Factor (EGF) of the species of the cells being cultured. These growth factors are commercially available from a variety of sources. Typically, these growth factors are purchased and reconstituted in appropriate liquid buffers, aliquoted and stored as per manufacturer's recommendations. Addition of bFGF to serum-free medium is known to promote proliferation of human M/ASCs. Growth factor-defined culture medium for human mesenchymal stem cells.

Holo-transferrin (Sigma, St. Louis, MO) of the species of the cells being cultured was added as a source for iron transport. Holo-transferrin, which transports iron, a trace mineral, is an indispensable requirement for cells as it is a cofactor for metabolic enzymes and enzymes involved in DNA synthesis. Cells acquire iron from carrier proteins such as holo-transferrin. In some embodiments, Bovine holo-transferrin is not used.

Adherence factors: adherence factors aid in attachment of cells to culture surfaces. These factors are largely unknown. Recently, Fetuin-A, a glycoprotein, was shown to be a major serum cell attachment factor. Fetuin-A (α2HS-Glycoprotein) is a major serum adhesive protein that mediates growth signaling in breast tumor cells. In some embodiments Fetuin from bovine serum (Sigma, St. Louis, MO) at 0.025% to 0.1% (w/v) to facilitate attachment of canine Ad-ASCs to culture surfaces. In some embodiments, Fetuin was not utilized.

Hormones affect growth and modulate the function of cells. There are both steroidal and non-steroidal hormones. Hydrocortisone (HCN), an adrenocortical hormone, assists in adhesion and growth of M/ASCs. FBS typically contains 100 nM to 1 μM HCN. In some embodiments, synthetic HCN is used to supplement the medium. In other embodiments, HCN of the species of the cells is used in the medium. Another hormone important for modulating the function of M/ASCs is progesterone. In some embodiments, synthetic progesterone is also added to the medium. In other embodiments, the progesterone used is specific to the species of the cells in the medium.

Insulin is important in many cellular functions, including glucose and amino acid uptake, lipogenesis, intracellular transport, and protein and nucleic acid synthesis. Enhanced glycolysis is a metabolic phenotype associated with undifferentiated state of M/ASC because it promotes glucose uptake and is supported by insulin. Species specific insulin, in a concentration of 0.17 μM, was added to the medium. In some embodiments, the insulin was derived from the species for which the cells were harvested. In some embodiments, the insulin was derived from a different species than the one from which the cells were harvested, where the insulin is compatible between the species.

Other components that assist M/ASC and other cell growth include buffering agents, antioxidants, and polyamines.

In addition to being a metabolic precursor, sodium bicarbonate, is commonly used as a buffer. Sodium bicarbonate was therefore added to the media in 2.4 g/L concentrations. Additional buffering capacity was added to the media on the form of HEPES, an organic buffer.

The diamine putrescine is a non-protein nitrogen base and the precursor for polyamines spermidine and spermine. Putrescine is associated with proliferation in several mammalian cell lines and important for maintenance of self-renewal in human embryonic stem (ES) cells. 56 μM of putrescine was added to the medium.

The cell culture medium is produced by beginning with DMEM (Dulbelco Modified Eagle Medium) and adding F-12. F-12 is a nutrient mixture to assist in cell growth and proliferation. Additional nutrients are added to the medium to create a medium for the growth of each species M/ASC's and islet cells.

The buffering system utilized in the medium is sodium bicarbonate, with HEPES to assist in the buffering process. In some embodiments of the invention the concentration of sodium bicarbonate is at least about 18 mM, e.g., at least about 24 mM, e.g., at least about 28 mM, e.g., at least about, e.g., at least about 32 mM, e.g., at least about 36 mM, e.g., at least about 40 mM.

In some embodiments of the invention the concentration of HEPES is at least about 1 mM, e.g., at least about 2 mM, e.g., at least about 3 mM, e.g., at least about 4 mM, e.g., at least about 5 mM, e.g., at least about 6 mM, e.g., at least about 7 mM.

As has been described above glutamine, particularly L-glutamine, is an important nutrient for cellular growth. GlutaMax™ (ThermoFisher) was chosen for the L-glutamine additive. GlutaMax™ is a dipeptide of L-glutamine and L-alanine. In some embodiments of the invention the concentration of L-glutamine is at least about 1.5 mM, e.g., at least about 2.0 mM, e.g., at least about 2.5 mM, e.g., at least about 3.0 mM, about 3.5 mM, e.g., at least about 4.0 mM. In some embodiments, the concentration of L-alanine is at least about 0.2 mM, at least about 0.4 mM, at least about 0.6 mM, at least about 0.8 mM, at least about 1.0 mM, at least about 1.2 mM.

In some embodiments, lipids are added to the media in a volume-to-volume concentration. In these embodiments, the lipid concentration added to the media is at least about 4%, e.g., at least about 6%, e.g., at least about 8%, e.g., at least about 10%, e.g., at least about 12%, e.g., at least about 14%, e.g., at least about 16%.

In some embodiments of the invention the concentration of insulin is at least about 0.10 μM, e.g., at least about 0.11 μM e.g., at least about 0.13 μM e.g., at least about 0.15 μM e.g., at least about 0.15 μM e.g., at least about 0.17 μM e.g., at least about 0.19 μM, e.g., at least about 0.21 μM e.g., at least about 0.23 μM e.g., at least about 0.25 μM.

In some embodiments of the invention the concentration of hydrocortisone is at least about 50 nM, e.g., at least about 60 nM e.g., at least about 80 nM e.g., at least about 90 nM e.g., at least about 100 nM e.g., at least about 110 nM e.g., at least about 120 nM, e.g., at least about 130 nM e.g., at least about 140 nM e.g., at least about 150 nM.

In some embodiments of the invention the concentration of progesterone is at least about 11 nM, e.g., at least about 13 nM e.g., at least about 15 nM e.g., at least about 17 nM e.g., at least about 19 nM e.g., at least about 21 nM e.g., at least about 23 nM, e.g., at least about 25 nM e.g., at least about 27 nM e.g., at least about 29 nM.

In some embodiments of the invention the concentration of putrescine is at least about 35 μM, e.g., at least about 40 μM e.g., at least about 45 μM e.g., at least about 50 μM e.g., at least about 56 μM e.g., at least about 60 μM e.g., at least about 65 μM, e.g., at least about 70 μM e.g., at least about 75 μM e.g., at least about 80 μM.

In some embodiments of the invention the concentration of Asc-2-p is at least about 100 μM, e.g., at least about 125 μM e.g., at least about 150 μM e.g., at least about 175 μM e.g., at least about 200 μM e.g., at least about 225 μM e.g., at least about 250 μM, e.g., at least about 275 μM e.g., at least about 300 μM.

In some embodiments of the invention the concentration of holo-transferrin is at least about 0.20 μM, e.g., at least about 0.25 μM e.g., at least about 0.30 μM e.g., at least about 0.35 μM e.g., at least about 0.40 μM e.g., at least about 0.45 μM e.g., at least about 0.50 μM, e.g., at least about 0.55 μM e.g., at least about 0.60 μM. In some embodiments, holo-transferrin is not added to the media.

In some embodiments of the invention the concentration of bFGF—for each species (for example bFGF-canine, bFGF-human, or bFGF-mouse) is at least about 1 ng/ml, e.g., at least about 2 ng/ml, e.g., at least about 3 ng/mL e.g., at least about 4 ng/ml, e.g., at least about 5 ng/ml, e.g., at least about 6 ng/ml, e.g., at least about 7 ng/ml, e.g., at least about 8 ng/ml, e.g., at least about 9 ng/ml, e.g., at least about 10 ng/ml, e.g., at least about 11 ng/mL, e.g., at least about 12 ng/ml, e.g., at least about 13 ng/mL.

In some embodiments of the invention the concentration of PDGF-BB for each species (for example canine or human) is at least about 1 ng/mL, e.g., at least about 2 ng/ml, e.g., at least about 3 ng/ml e.g., at least about 4 ng/ml, e.g., at least about 5 ng/ml, e.g., at least about 6 ng/ml, e.g., at least about 7 ng/ml, e.g., at least about 8 ng/ml, e.g., at least about 9 ng/ml, e.g., at least about 10 ng/ml, e.g., at least about 11 ng/ml, e.g., at least about 12 ng/mL, e.g., at least about 13 ng/mL.

In some embodiments of the invention the concentration of EGF (for example EGF canine or EGF human) is at least about 1 ng/mL, e.g., at least about 2 ng/ml, e.g., at least about 3 ng/ml e.g., at least about 4 ng/mL, e.g., at least about 5 ng/ml, e.g., at least about 6 ng/ml, e.g., at least about 7 ng/ml, e.g., at least about 8 ng/ml, e.g., at least about 9 ng/mL, e.g., at least about 10 ng/ml, e.g., at least about 11 ng/mL, e.g., at least about 12 ng/ml, e.g., at least about 13 ng/mL.

In some embodiments of the invention the concentration of TGF-β1 is at least about 0.25 ng/ml, e.g., at least about 0.5 ng/ml, e.g., at least about 0.75 ng/ml e.g., at least about 1.0 ng/ml, e.g., at least about 1.25 ng/ml, e.g., at least about 1.50 ng/ml, e.g., at least about 1.75 ng/ml, e.g., at least about 2.0 ng/mL. In some embodiments, TGF-β1 is not utilized in the medium.

Albumin and other serum proteins may be included in embodiments. In some embodiments of the invention the concentration of canine Serum is at least about 1%, e.g., at least about 2%, e.g., at least about 3%, e.g., at least about 4%, e.g., at least about 5%, e.g., at least about 6%, e.g., at least about 7%, e.g., at least about 8%, e.g., at least about 9%, e.g., at least about 10%, e.g., at least about 11%, e.g., at least about 15%.

In some embodiments of the invention the concentration of SA (species specific serum albumin-see comment associated with paragraph 60) is at least about 0.001 g/L, e.g., at least about 0.002 g/L, e.g., at least about 0.003 g/L e.g., at least about 0.004 g/L, e.g., at least about 0.005 g/L, e.g., at least about 0.006 g/L, e.g., at least about 0.007 g/L, e.g., at least about 0.008 g/L, e.g., at least about 0.009 g/L, e.g., at least about 0.010 g/L In some embodiments, SA is absent from the media.

In some embodiments of the invention the concentration of Fetuin is at least about e.g., at least about 0.25 ng/ml, e.g., at least about 0.50 ng/mL, e.g., at least about 0.75 ng/ml, 1 g/mL, e.g. at least about 2 g/mL, e.g., at least about 3 g/mL e.g., at least about 4 g/mL, e.g., at least about 5 g/mL, e.g., at least about 6 ng/ml, e.g., at least about 7 ng/ml, e.g., at least about 8 ng/ml, e.g., at least about 9 ng/ml, e.g., at least about 10 ng/mL, e.g., at least about 11 ng/ml, e.g., at least about 12 ng/ml, e.g., at least about 13 ng/mL. In some embodiments, Fetuin is absent from the media.

### Example 1 Canine Specific Media

Dogs are increasingly the recipient of cellular therapeutics, particularly Mesenchymal Stromal Cells (MSCs), for the treatments of various disorders, including renal diseases, bone and joint disorders, diabetes, and immune disease. These therapies require isolation, purification and often culture of MSCs over many doublings. Such culture requires the use of serum supplemented culture medium and given the susceptibility of dogs to foreign protein reactions, ideally, the serum-supplement is canine-derived so that cells that are introduced into the dogs are not carriers of xenogeneic proteins that might cause an antigenic response in the recipient. A xeno-free, reduced serum culture medium (SCT255c) has been developed for culture of canine cells. In order to prevent xenogeneic immune responses, this medium contains only canine-derived proteins, and canine serum, but no other animal proteins. Two cell types; Adipose-derived or bone marrow-derived Mesenchymal Stromal Cells (A/MSCs), and pancreatic Islet Cells (ICs), have been tested using this medium vs standard, commercially available culture media. A/MSCs cultured in SCT255c show faster growth rates and higher yields, and also have a superior immune modulatory profile compared to those cultured in standard, 10% canine serum containing medium. For cells derived from the canine Islets of Langerhans, culture in SCT255c allows greater doubling potential, superior growth rates, and preserved islet endocrine gene expression over culture in standard growth media. This medium requires no attachment factors such as Fetuin A, which were shown by others to be required for support of canine MSC culture. Further, serum need for culture of M/ASCs and ICs is greatly reduced to 1% for M/ASCs and P0 ICs, and 5% for primary Islets, decreasing both the ethical burden and the monetary cost of culturing cells. See Tables 1 and 2.

SCT's reduced serum medium for canine M/ASCs and islet cells was compared head-to-head with standard culture media, either DMEM/F-12+10% (v/v) canine serum, or low glucose DMEM+10% (v/v) canine serum. Cells were assessed for morphology, growth rates, and gene expression profiles of various beneficial cytokines.

Several variations of the reduced serum have been tested. The composition of the most effective variant of the reduced serum medium is described in Table 1. In this variation the bFGF is 2 ng/ml, the PDGF-BB is 5 ng/ml, and the EGF is 5 ng/ml. This variation was named SCT255 and will be referred to as such going forward.

TABLE 1

| | | Final | |
|---|---|---|---|
| Type | Component | Concentration | |
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (porcine; | 0.17 | uM |
| | identical to canine) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF canine | 2 | ng/ml |
| | PDGF-BB canine | 5 | ng/ml |
| | EGF-canine | 5 | ng/ml |
| Protein/osmotic | Canine Serum | 1% | v/v |

Alternative formulations of a reduced serum medium were also tested, and these compositions are also included as Table 2 and Table 3.

TABLE 2

| | | Final | |
|---|---|---|---|
| Type | Component | Concentration | |
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine (200 mM) | 3.25 | mM |
| | L-alanine | 0.8 | mM |
| Lipids | Lipid Concentrate | 0.10% | v/v |
| Hormones | Insulin (porcine; | 0.17 | uM |
| | identical to canine) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF canine | 2 | ng/ml |
| | PDGF-BB canine | 2 | ng/ml |
| | EGF-canine | 2 | ng/ml |
| Protein/osmotic | Canine Serum | 1% | v/v |

TABLE 3

| | | Final | |
|---|---|---|---|
| Type | Component | Concentration | |
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (porcine; | 0.17 | uM |
| | identical to canine) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF canine | 10 | ng/ml |
| | PDGF-BB canine | 5 | ng/ml |
| | EGF-canine | 10 | ng/ml |
| Protein/osmotic | Canine Serum | 1% | v/v |

### Cells Assessed

### Canine M/ASCs

MSCs isolated from sub-cutaneous fat (aka ASCs) from 6 dogs were examined. Donor dogs varied in age, were of either gender, and were either healthy or had heart failure (atrial fibrillation or ventricular tachycardia) of 6 weeks to 2 years duration at the time of fat donation (see Table 4).

TABLE 4

| Donor number | Gender | Age at donation | | Health |
|---|---|---|---|---|
| 1 | F | 1 | year | Healthy |
| 2 | F | 1 | year | Heart failure |
| 3 | M | 1 | year | Healthy |
| 4 | M | 1 | year | Heart failure (x6 weeks) |
| 5 | F | 4 | years | Heart failure (x2 years) |
| 6 | F | 4 | years | Heart failure (x2 years) |

### Cell Growth

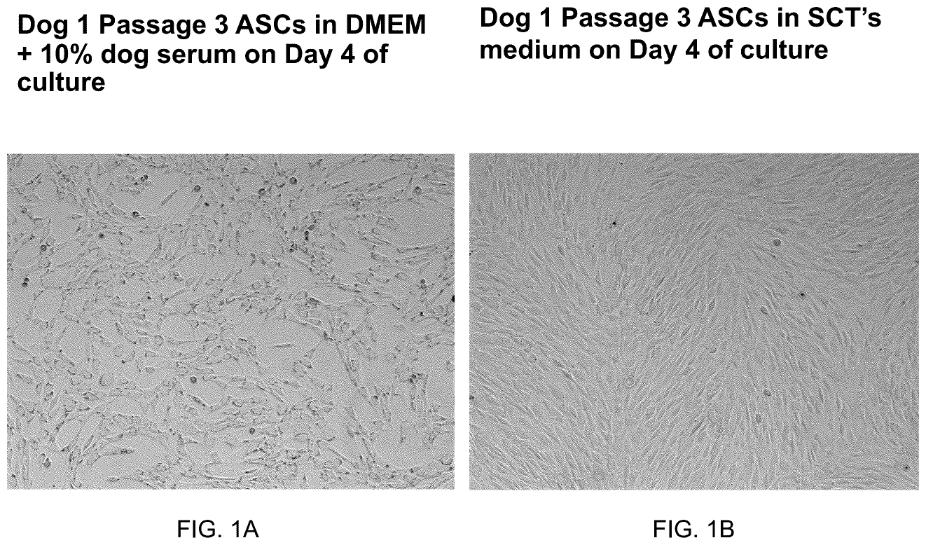

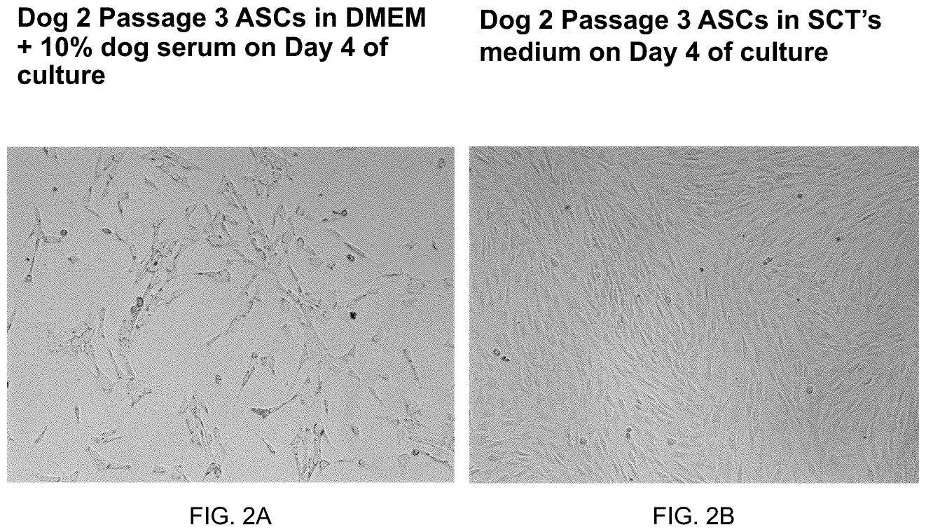

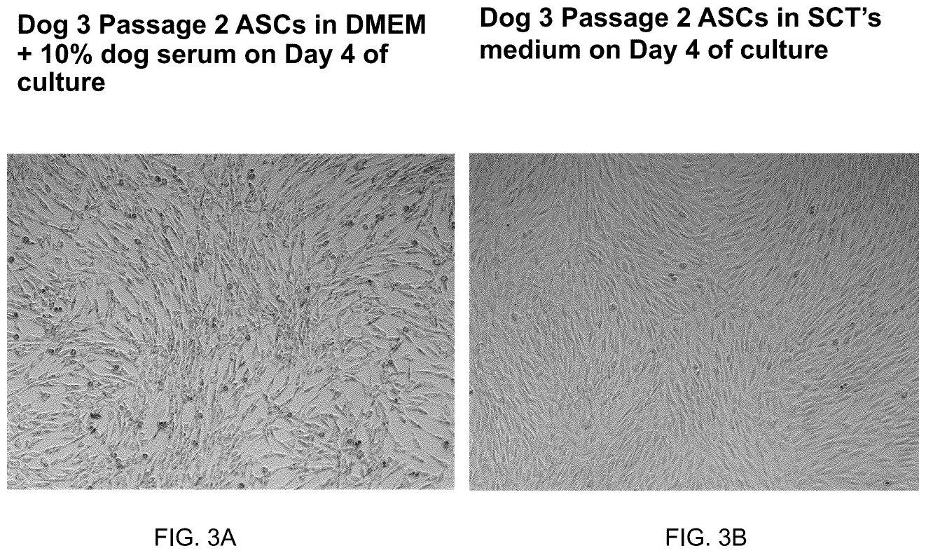

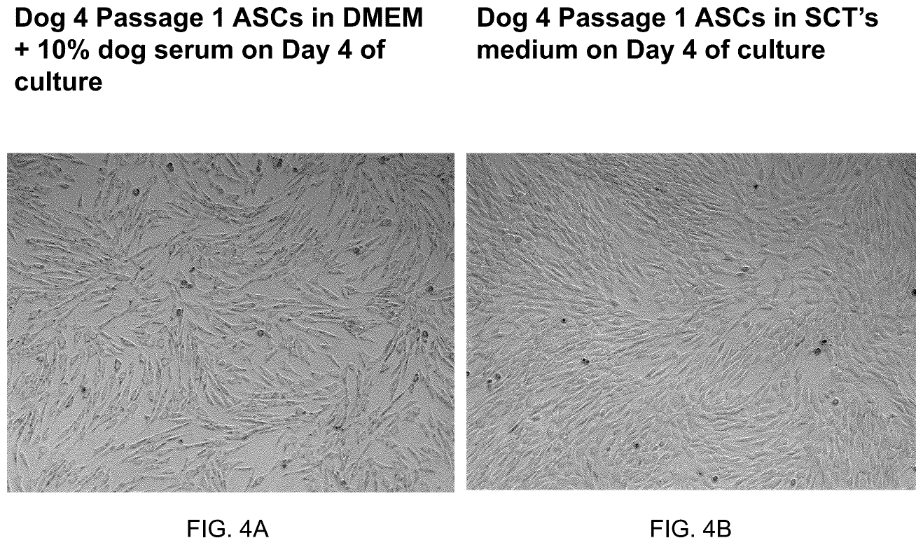

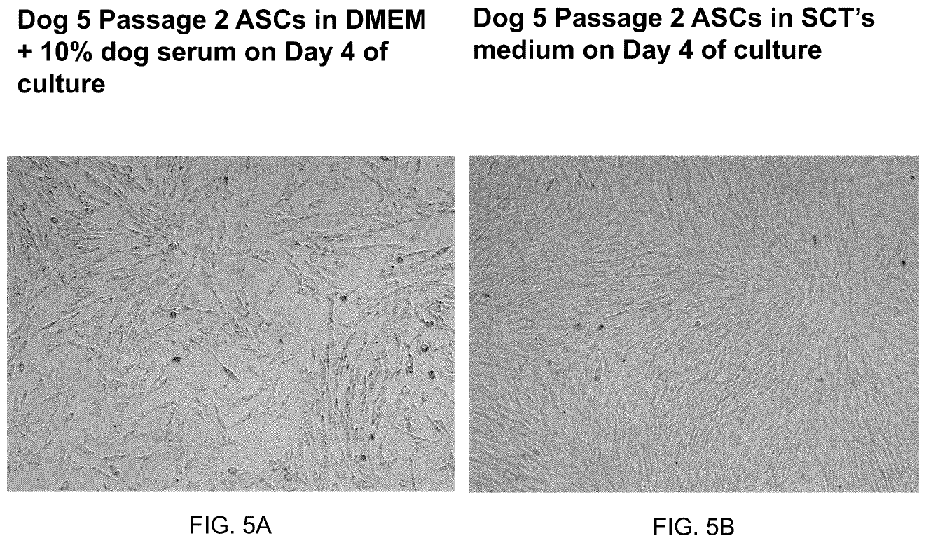

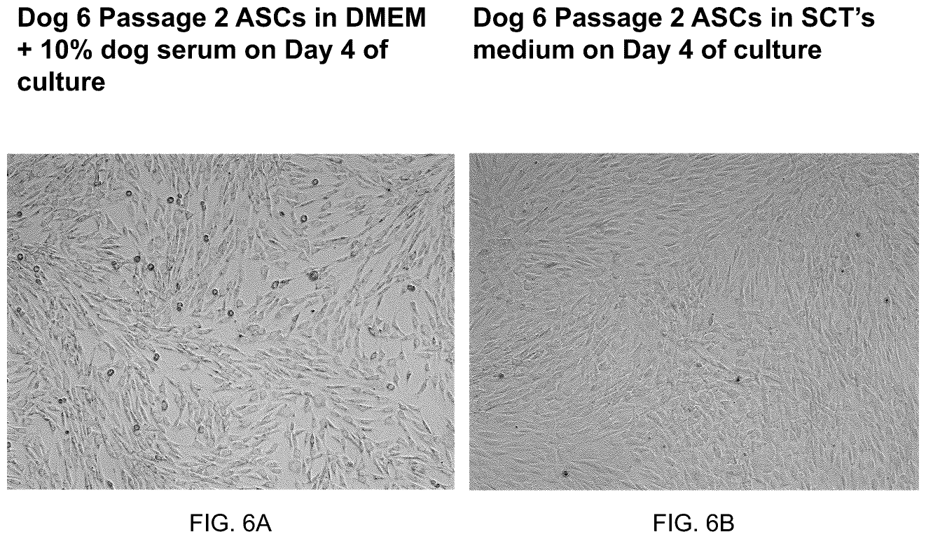

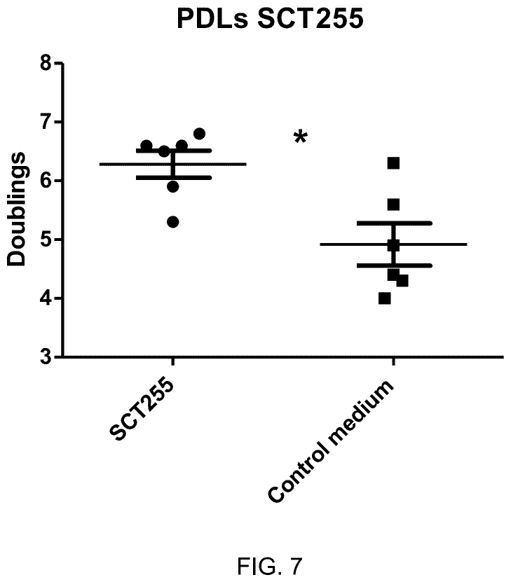

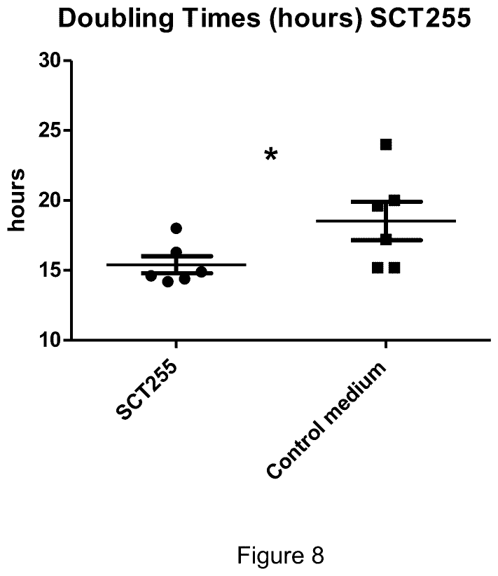

As shown in FIGS. 1 through 6, M/ASCs from all tested dogs exhibit a more cobblestone appearance when cultured in SCT's formulation than standard culture medium. Canine M/ASCs cultured in standard culture medium (DMEM or DMEM low glucose)+10% (v/v) canine serum for 4 days underwent a mean of 4.9 population doublings. The same canine M/ASCs cultured in SCT's medium, by contrast underwent statistically significantly higher, 6.2 doublings (FIG. 7) in the same time frame due to significantly higher doubling times of cells cultured in SCT's medium (FIG. 8).

### Propagation

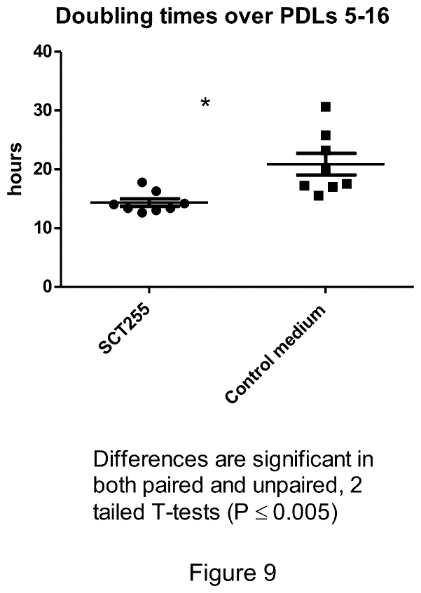

Canine M/ASCs from the donors listed in Table 3 were cultured for 4 to 6 passages in either SCT's formulation or standard culture medium. The faster doubling times and consequently increased number of PDLs observed for cells cultured in SCT's formulation and shown in FIGS. 7 and 8 were maintained through 16 doublings (see FIG. 9). Indeed, doubling times were significantly shorter and more consistent for canine M/ASCs cultured in SCT255 than for cells cultured in standard media+10% (v/v) serum (FIG. 9). FIGS. 7-9 indicate that variation introduced by cell differences, often a result of different donors is reduced when cells are cultured in this medium. It is often desirable to produce cells that have uniform characteristics, this give more consistent results both in testing and in use of cells such as in a cellular therapeutic product.

### Identity

Canine M/ASCs are in part identified by their expression of cell surface markers, with positive expression of CD90 and CD44, and the absence of expression of CD34, CD45 and Class II antigens (DLA-DR). As shown in Table 5, Canine M/ASCs cultured in SCT's medium display positive expression of CD90 and CD44, but are negative for expression of CD34, CD45, and DLA-DR. Expression of DLA-DR after overnight exposure to INF-γ (10 ng/ml) was also assessed by FACS and found to be negative.

Passaging of cells to ≥20 PDLS did not affect expression of these markers (see Table 5).

TABLE 5

| Cells | | | | | | | | (+INF-g) |
|---|---|---|---|---|---|---|---|---|
| from Dog | Passage | Doublings | CD34 (−) | CD45 (−) | CD44 (+) | CD90 (*) | DLA-DR | DLA-DR |
| 1 | 2 | 7.6 | 3.6 | 0 | 100 | 100 | 0.2 | 0.4 |
| | 4 | 19.5 | 0.4 | 0 | 95 | 99.8 | 0.3 | 0.3 |
| 2 | 2 | 10 | 0.7 | 0 | 100 | 98.2 | 0.1 | 0.4 |
| | 4 | 21.6 | 0 | 0 | 100 | 98.5 | 4 | 0.8 |
| 3 | 2 | 5 | 0 | 0 | 97 | 99.9 | 1.14 | 4 |
| | 4 | 20.6 | 0 | 0 | 99.1 | 99.8 | 0.088 | 0.23 |
| 4 | 2 | 5.7 | 0 | 0.52 | 93 | 98.1 | 0 | 2 |
| | 6 | 24.9 | 0 | 0 | 99 | 99 | 0 | 0 |
| 5 | 2 | 7 | 0.2 | 0 | 95 | 95 | 0.2 | 0.1 |
| | 4 | 23.2 | 2.1 | 0 | 99 | 99 | 0.2 | 0.1 |
| 6 | 2 | 7.1 | 0 | 0.06 | 99.4 | 100 | 0.12 | 2.08 |
| | 4 | 18 | 0 | 0 | 97.9 | 99.9 | 0.21 | 0.67 |

### Gene Expression

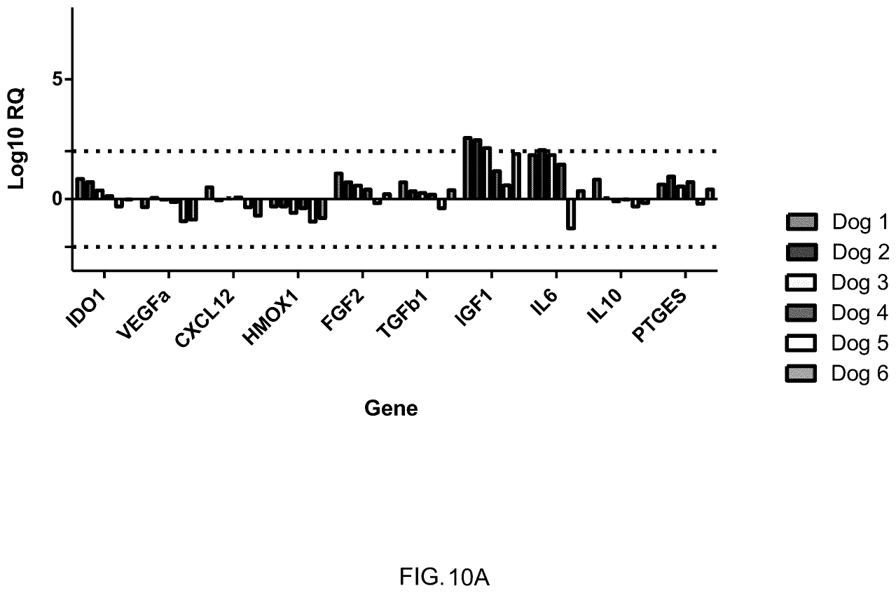

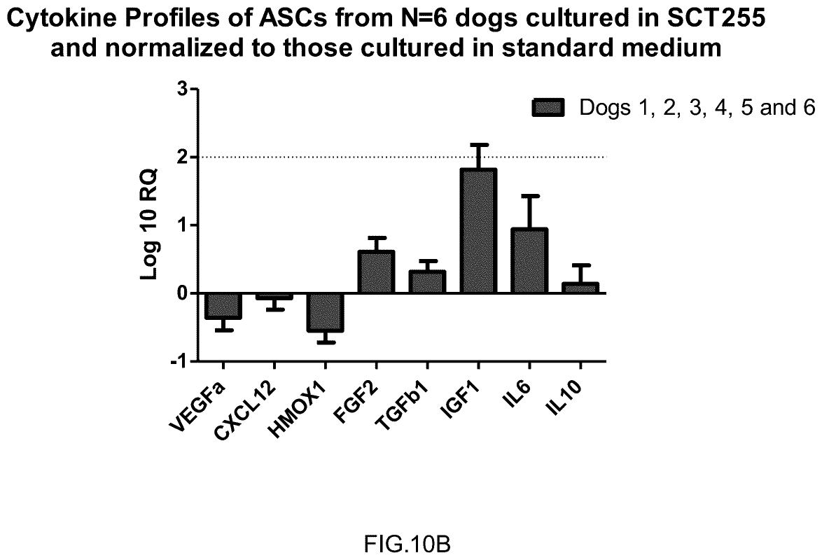

Gene expression of beneficial, paracrine cytokines of M/ASCs cultured in SCT's medium vs. those of the same cells cultured in standard, 10% (v/v) serum containing medium are similar (FIGS. 10A-10B). Expression levels of IFG-1 and IL-6, both involved in immune-modulation by MSCs, trend higher in cells cultured in SCT's medium than in normal medium, but do not reach statistically significant levels (log 10[RQ]≥±2). IGF-1 expression by MSCs has pro-regenerative and immune modulatory actions, and IL-6 is also immune modulatory.

### Immune Modulation

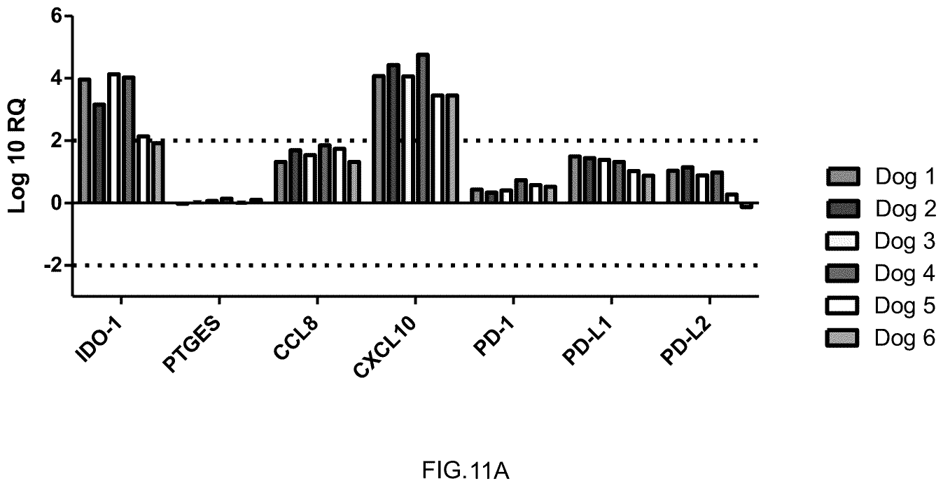

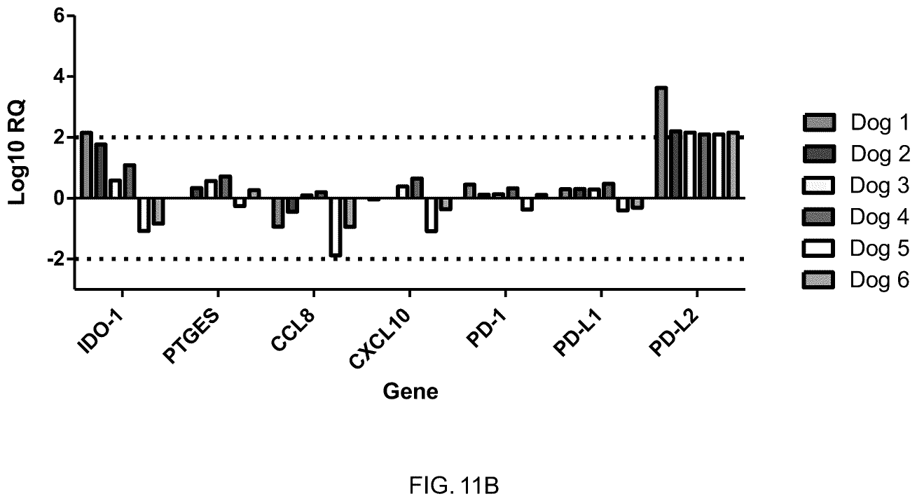

As shown in FIGS. 11A-11B, cells cultured in SCT's medium respond to overnight INF-exposure by inducing expression of IDO-1 and other genes involved in immune modulation. Culture in SCT255 results in the consistent and highly significant up-regulation of the powerful anti-inflammatory cytokine CXCL10.

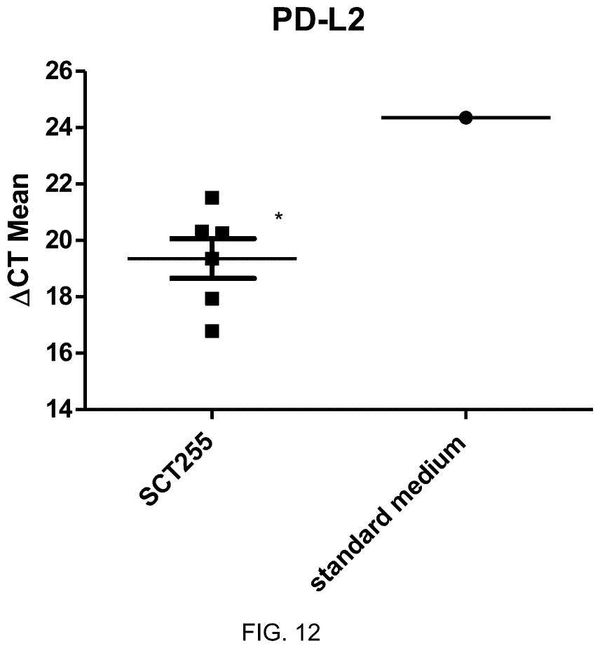

Unexpectedly, PD-L2, a ligand of PD-1 which with PD-L1 confers immune tolerance is expressed in canine M/ASCs cultured in SCT's medium even in the absence of INF-γ exposure. This immune modulatory gene is frequently not expressed when cells are cultured in standard medium without INF-γ (FIG. 12). Furthermore, induction of PD-L2 upon overnight exposure of cells to INF-γ is significantly stronger when the cells have been cultured in SCT's medium vs. standard culture media (either DMEM or DMEM low glucose; FIG. 11B).

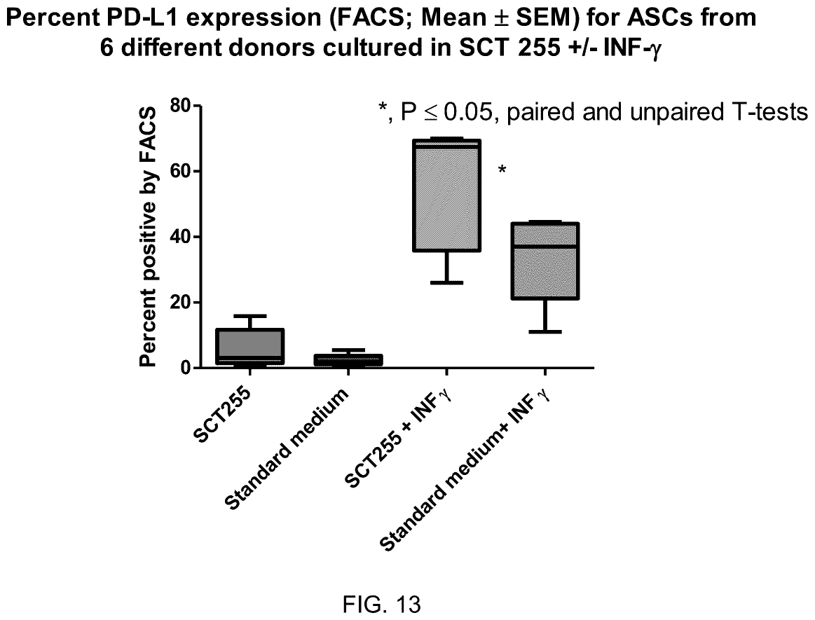

Secretion of PD-L1 and PD-L2 by human M/ASCs has been shown to directly affect T cell behavior, induce immune tolerance, and mediate antifibrotic effects. As shown in FIG. 13, upon exposure to INF-γ, PD-L1 is significantly more highly expressed in cells cultured in SCT 255 than in standard medium. PD-L1 expression by cancer cells is known to induce tolerance and allow such cells to evade destruction by the immune system. Interruption of the PD-1/PD-L1 pathway is currently used as an anti-cancer therapeutic in humans and is being developed as one in canines due to similarities of this pathway among the two species. Thus it is likely that culture of canine M/ASCs in SCT's medium under standard conditions will result in cells with better immune modulatory capabilities than those cultured using standard culture media.

### Canine Islet Cells

Islets isolated from pancreata from 6 dogs were cultured and assessed. Donor dogs varied in age, were of either gender, and were either healthy or had heart failure of 6 weeks to 2 years duration at the time of pancreas donation (see Table 6).

TABLE 6

| | | | | Warm | Cold |
|---|---|---|---|---|---|
| | | Age at | | ischemia | ischemia |
| Donor | | donation | | time | time |
| number | Gender | (years) | Health | (min) | (min) |
| 7 | M | 1.5 | Heart failure | 4 | 26 |
| 8 | M | 1.5 | Heart failure | 11 | 24 |
| 9 | F | 6 | Heart failure | 6 | 43 |
| 10 | F | 1 | Healthy | 6 | 41 |
| 11 | F | 3 | Heart failure | 7 | 22 |
| 12 | M | 2 | Heart failure | 5 | 25 |

### Cell Growth and Propagation

Islets were isolated and cultured in either SCT's medium formulation or in standard culture medium (DMEM−low glucose formulation+20% (v/v) canine Serum) as previously described. Because islet cells require more serum in their culture medium, SCT's medium for culture of primary islets was formulated with a serum concentration of 5% (v/v) rather than 1% (v/v) required for A/MSCs.

P0 islet cells were harvested from such cultures when ~90% confluent using 2× Trypsin EDTA, and assessed for yields, growth rates, and doublings, and then passaged to P1. P1 cultures were likewise cultured, harvested, and assessed, and performance differences between the SCT255 and standard media were determined.

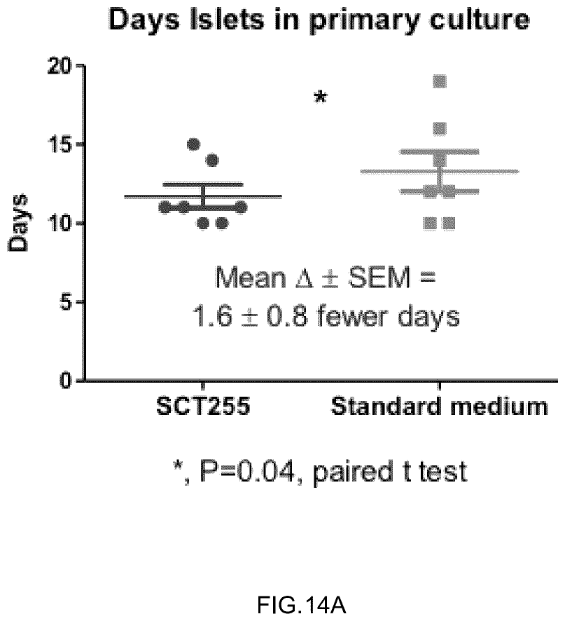

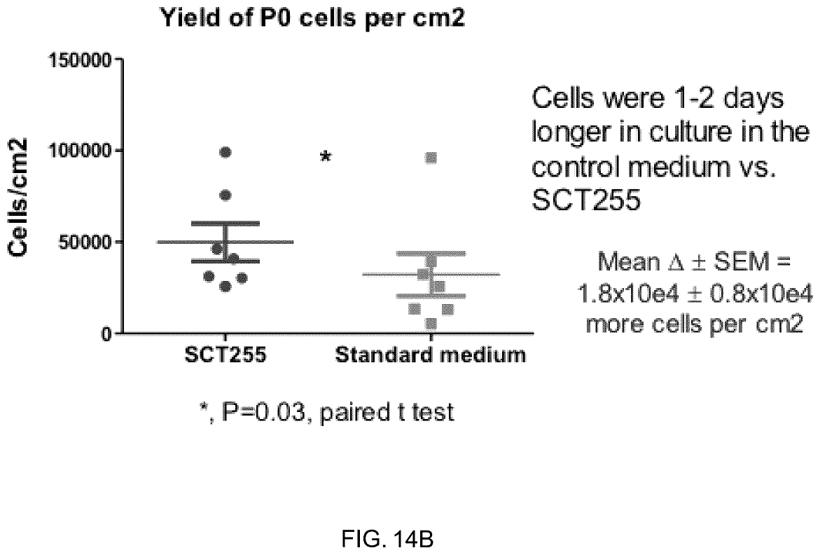

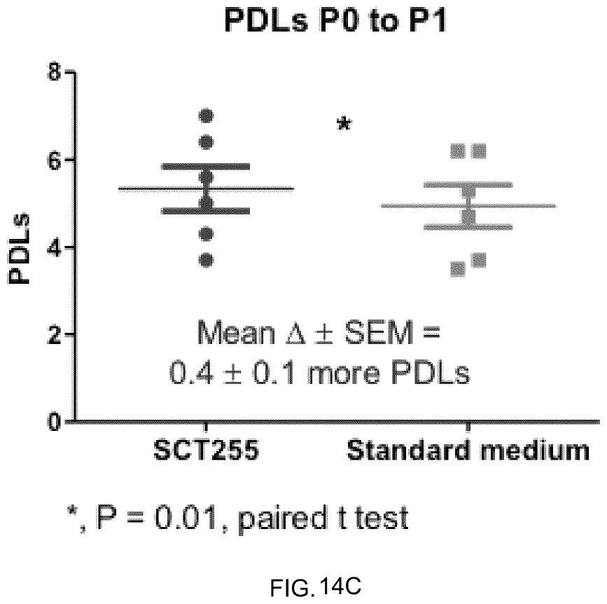

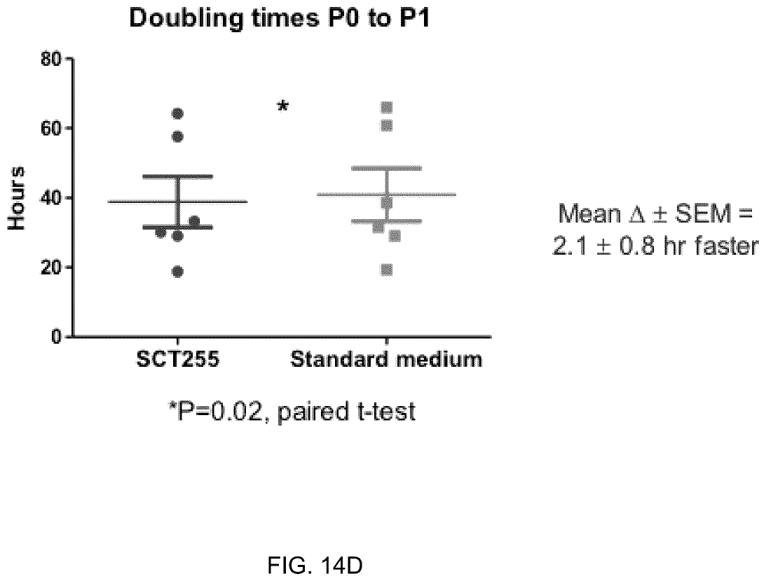

As shown in FIGS. 14A-14D, SCT's medium consistently supported significantly superior and more rapid outgrowth of primary islets, yielding a mean of ~20,000 more P0 islet cells (primary culture) per cm2 in a mean of ~2 fewer days than standard culture medium (FIGS. 14A and B). Similarly, doubling times were a mean of 2 hours faster when P0 cells were passaged and cultured to P1 (FIGS. 14 C and D).

### Gene Expression

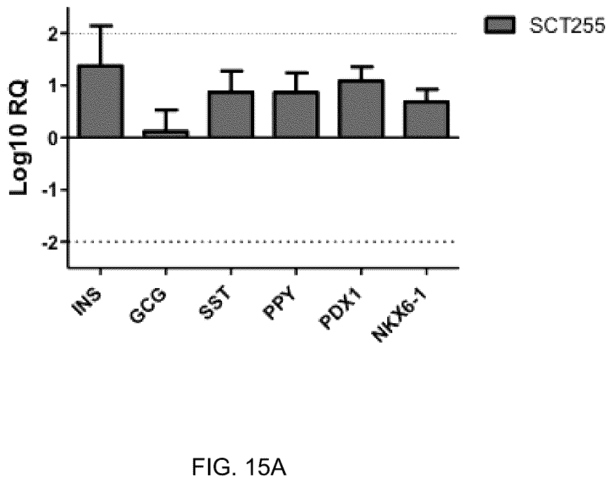

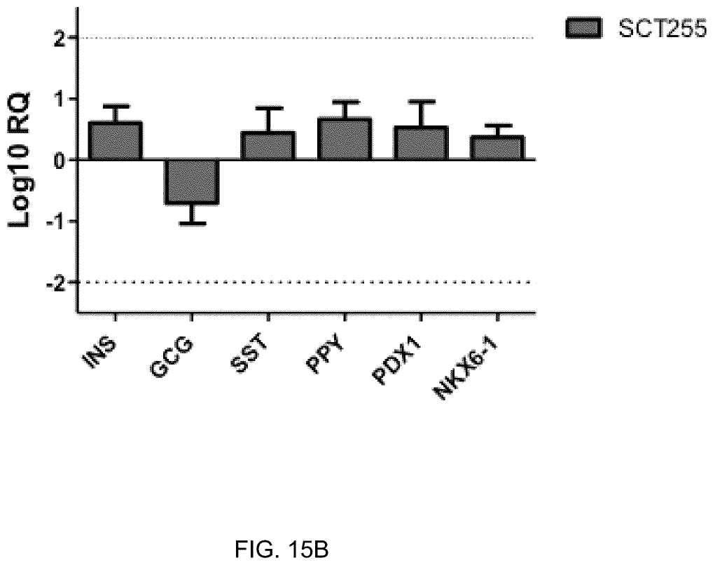

For P0 and P1 islet cells cultured in either SCT's medium formulation or standard culture medium, endocrine gene expression was assessed by rtPCR. Results for SCT's medium were normalized to those of the same passage of cells cultured in standard culture medium using the delta-delta ct method, where log 10[RQ]≥±2 is considered statistically significant. As shown in FIGS. 15A-15B, although islet cells cultured in SCT's medium had undergone more doublings, endocrine gene expression was well preserved, and for all assessed genes except glucagon at P1 (FIG. 15B), trended toward increased expression levels.

### Exosomes

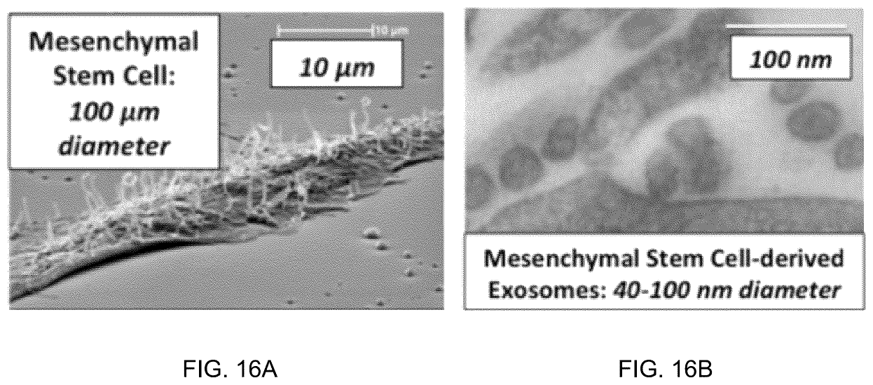

Cell-derived extracellular vesicles such as exosomes from MSCs are increasingly envisioned as a potential therapeutic for situations where the cells' therapeutic cargo is desired, but the microvasculature is compromised such that the cells themselves would potentially further compromise or occlude it due to their larger size (FIGS. 16A and 16B).

In order to determine whether the SCT 255c base medium (SCT 255c without a serum additive) is suitable for collecting and harvesting exosomes, and whether the exosomes derived from this medium are comparable to or different from those derived from cells incubated in DMEM (standard base medium), the following experiment was conducted.

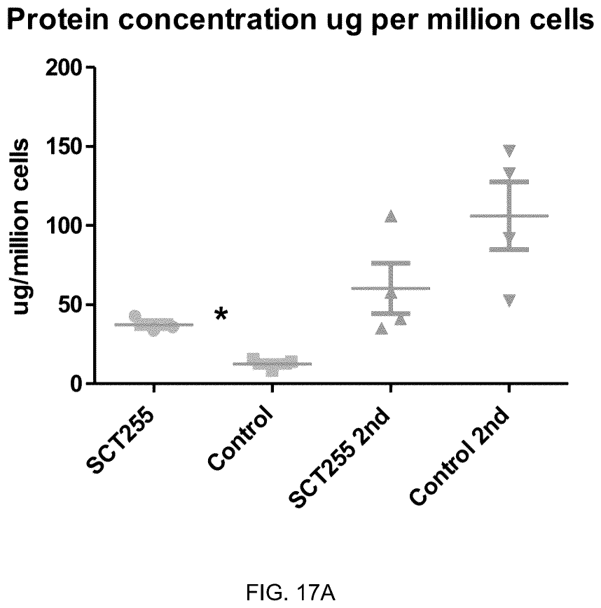

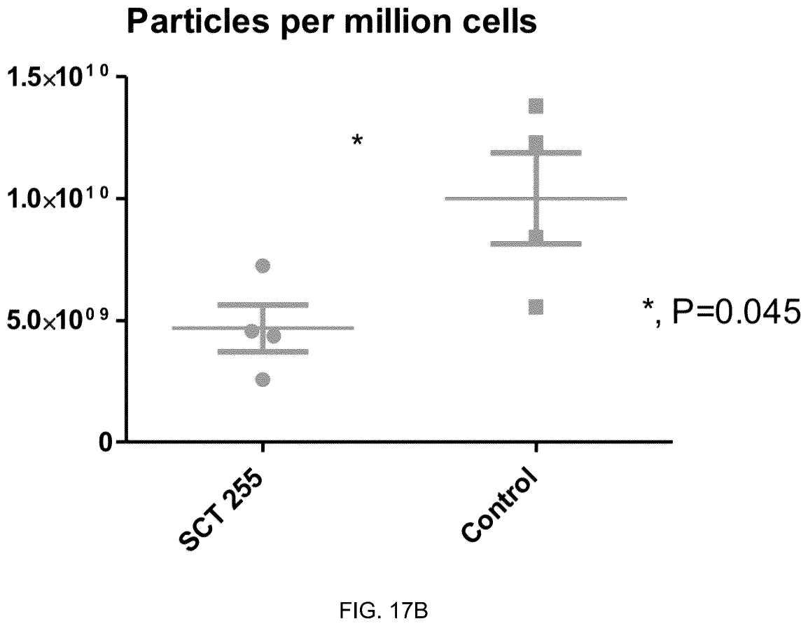

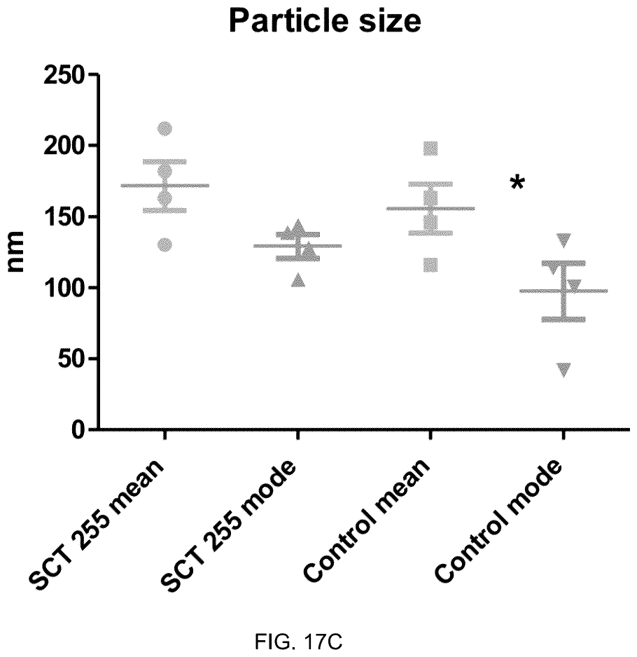

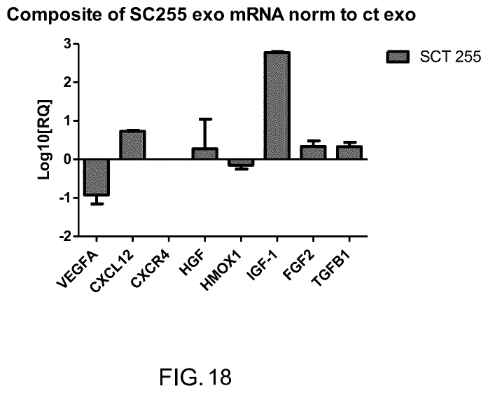

ASCs from dogs 1, 2, 3 and 4 were cultured to 85% confluence in T75 flasks using either DMEM+10% (v/v) dog serum or SCT 255c+1% (v/v) dog serum. Once cells reached 85% confluence, the medium for both sets of cells was replaced with base medium (no serum), and cells were cultured an additional 24 hours, after which, medium was collected, and cells were harvested and counted. Exosomes from each set of medium were isolated using the Exoquick TC kit. Exosomes from each set were analyzed for protein content using the Bradford assay, relevant mRNA cargo via rtPCR, as well as for size and particle number using Nanosite. Consistent with previous findings reported above, cells grew significantly faster and underwent significantly more PDLs in SCT255c than in standard culture medium. Cells cultured in SCT255c did yield more consistent results for protein concentration and mode particle size than did cells cultured in standard medium (FIGS. 17A-17B). Sizes and particle numbers of microvesicles collected from SCT255c vs standard medium cultured MSCs were not significantly different from each other (FIG. 17C). Inconsistent yields and quality of extracellular vesicles has been seen as one of the barriers to therapeutic use of MSC-derived exosomes to date. Thus, SCT255c base medium may offer a significant advantage for extracellular vesicle isolation over other base media. With the exception of IGF-1, mRNA cargo was not significantly different in the exosomes derived from cells cultured in SCT255c vs control medium (FIG. 18), but again, reflecting the potentially superior immune modulating capacity of MSCs and their extracellular vesicles when cultured in this vs other media formulations.

### Example 2 Human Specific Growth Media

The serum-supplement is human-derived so that cells that are introduced into the humans are not carriers of xenogeneic proteins that might cause an antigenic response in the recipient. A xeno-free, reduced serum culture medium (SCT255h) was developed for culture of human cells. In order to prevent xenogeneic immune responses, this medium contains only human-derived proteins, and human serum, but no other animal proteins. Two cell types; Adipose-derived or bone marrow-derived Mesenchymal Stromal Cells (A/MSCs), and pancreatic Islet Cells (ICs), are undergoing testing using this medium vs standard, commercially available culture media.

The variants of the reduced serum medium for human M/ASCs and islet cells were compared head-to-head with standard culture media. Cells will be assessed for morphology, growth rates, and gene expression profiles of various beneficial cytokines.

Several variations of the reduced serum medium have been produced and tested. Table 7 depicts the human specific serum equivalent to the most effective canine serum.

TABLE 7

| Type | Component | Final | |
|---|---|---|---|
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (porcine; | 0.17 | uM |
| | identical to human) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF human | 2 | ng/ml |
| | PDGF-BB human | 5 | ng/ml |
| | EGF-human | 5 | ng/ml |
| Protein/osmotic | Human Serum or platelet lysate | 1% | v/v |

TABLE 8

| Type | Component | Final | |
|---|---|---|---|
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (porcine; | 0.17 | uM |
| | identical to Human) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF human | 10 | ng/ml |
| | PDGF-BB human | 5 | ng/ml |
| | EGF-human | 10 | ng/ml |
| Protein/osmotic | Human Serum or platelet lysate | 2% | v/v |

### Cells Assessed

### Human M/ASCs

[1] Human MSCs derived from bone marrow were cultured in SCT255 h and 1% (v/v) human Platelet Lysate (hPLA) and compared to bone marrow-derived MSCs cultured in alpha MEM+10% (v/v) hPLA which was used as a control. Additional variations of the cell media were also assessed, one such variation utilized SCT255h+2% (v/v) hPLA. This variation of the Medium had similar results to the SCT 255h+1% (v/v) hPLA.

[2] Two additional MSC lines were also assessed. The cell lines were hMSC purchased from RoosterBio P1. These are called “Rooster”, and hMSC-041508-P2, from a previous clinical trial. These are called 041508.

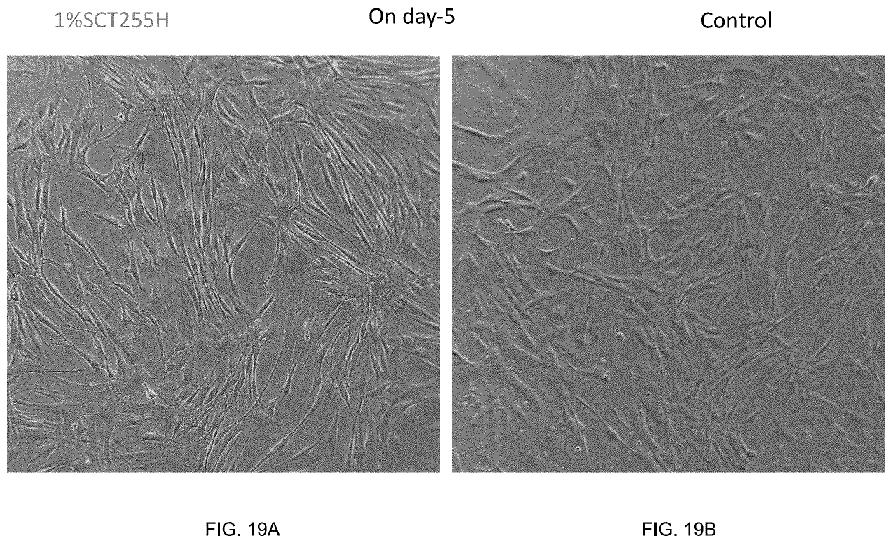

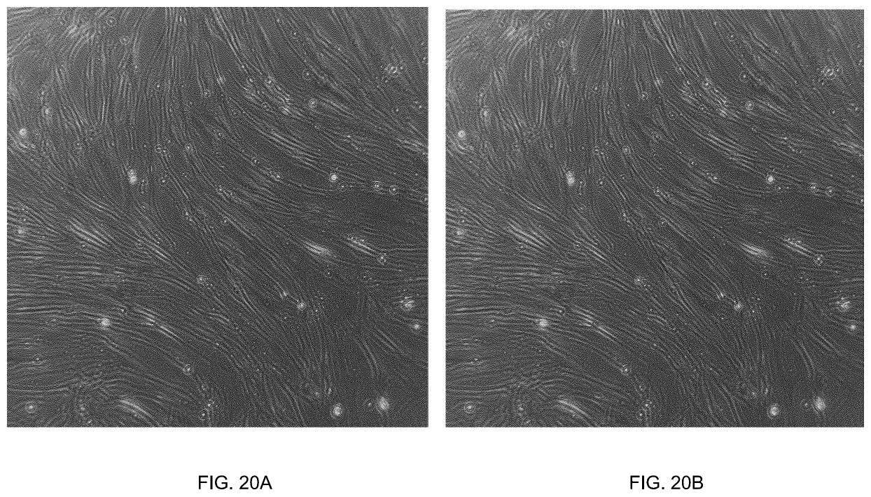

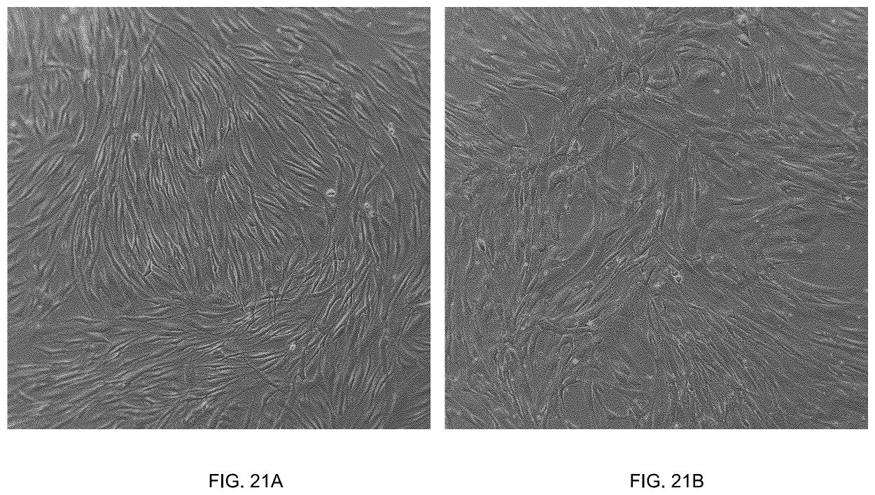

### Cell Growth

[3] There was little difference in the growth rates and cell morphologies of the cells grown in the SCT255 h medium and the control medium. FIG. 19A shows the bone marrow derived MSCs in the SCT255h+1% (v/v) hPLA medium and FIG. 19B shows the same cells in the control media. As can be seen in the micrographs the cells in both media appear similar, a slight difference is that the cells cultured in the SCT255 h medium are longer and spindlier. The additional cell lines also show little difference between the cells grown in the SCT255 h medium and the control. FIG. 20A is a micrograph of the 041508 cells grown in the SCT255 h medium, and FIG. 20B is the 041508 cells grown in the control medium. FIG. 21A is a micrograph of the Rooster cells grown in the SCT255h medium, and FIG. 21B is the Rooster cells grown in the control medium.

### Propagation

[4] The cells in both media underwent 3.8 doublings and each doubling time was about 37 hours. Table 9 shows the Rooster and 041508 cell lines as cultured in the various media.

TABLE 9

| Test 1% (v/v) | | | | | | | | |
|---|---|---|---|---|---|---|---|---|
| hPLA/SCT255H | | | | | | | | |
| using | | | Days | | | | | |
| hMSC w/2xTrypLE | Passage | seeding/ | in | cells/flask | Confluency | | | Doubling |
| or trypsin | # | flask | Culture | @ harvest | % | Fold | PDL | time (hr) |
| 1% (v/v) | 2 | 2.50E+04 | 5 | 6.00E+05 | 90% | 24.00 | 4.6 | 26.2 |
| SCT255H-Rooster- | | | | | | | | |
| p2 | | | | | | | | |
| 1% (v/v) | 2 | 2.50E+04 | 5 | 6.20E+05 | 90% | 24.80 | 4.6 | 25.9 |
| SCT255H + INFr- | | | | | | | | |
| Rooster-p2 w/2x | | | | | | | | |
| TrypLE | | | | | | | | |
| 1% (v/v) | 2 | 2.50E+04 | 5 | 6.10E+05 | 90% | 24.40 | 4.6 | 26.0 |
| SCT255H + INFr- | | | | | | | | |
| Rooster-p2 | | | | | | | | |
| w/1xTrypsin | | | | | | | | |
| 10% (v/v) hPLA/a- | 2 | 2.50E+04 | 5 | 6.60E+05 | 80% | 26.40 | 4.7 | 25.4 |
| MEM-Rooster-p2 | | | | | | | | |
| 1% (v/v) | 3 | 2.50E+04 | 7 | 2.30E+05 | 85% | 9.20 | 3.2 | 52.5 |
| SCT255H-041508- | | | | | | | | |
| P3 w/1xTrypsin | | | | | | | | |
| 1% (v/v) | 3 | 2.50E+04 | 7 | 2.40E+05 | 85% | 9.60 | 3.3 | 51.5 |
| SCT255H + INFr- | | | | | | | | |
| 041508-P3 | | | | | | | | |
| w/1xTrypsin | | | | | | | | |
| 10% (v/v) hPLA/a- | 3 | 2.50E+04 | 7 | 2.40E+05 | 85% | 9.60 | 3.3 | 51.5 |
| MEM-041508-P3 | | | | | | | | |
| w/1xTrypsin | | | | | | | | |

### Identity

[5] Human MSCs are in part identified by their expression of cell surface markers. The cells from both culture media types did not have significantly different expression of the cell surface markers.

[6] Passaging of cells to >20 PDLS is not expected to affect expression of these markers (see Table 5).

### Gene Expression

[7] Gene expression of beneficial, paracrine cytokines of MSCs cultured in SCT's medium vs. those of the same cells cultured in standard, 10% (v/v) serum containing medium are anticipated to be similar. Expression levels of IFG-1 and IL-6, both involved in immune-modulation by MSCs, is expected to trend higher in cells cultured in SCT's medium than in normal medium, but do not reach statistically significant levels (log 10[RQ]≥±2). IGF-1 expression by MSCs has pro-regenerative and immune modulatory actions, and IL-6 is also immune modulatory.

### Immune Modulation

[8] Cells cultured in SCT's medium are anticipated to respond to overnight INF-γ exposure by inducing expression of IDO-1 and other genes involved in immune modulation (note: induction of CCL8 and CXCL10 has been published for human but not canine MSCs). Culture in SCT255 results in the consistent and highly significant up-regulation of the powerful anti-inflammatory cytokine CXCL10.

[9] Secretion of PD-L1 and PD-L2 by human MSCs has been shown to directly affect T cell behavior, induce immune tolerance, and mediate antifibrotic effects. upon exposure to INF-γ, PD-L1 is expected to be more highly expressed in cells cultured in SCT 255 than in standard medium. PD-L1 expression by cancer cells is known to induce tolerance and allow such cells to evade destruction by the immune system. Interruption of the PD-1/PD-L1 pathway is currently used as an anti-cancer therapeutic in humans.

### Human Islet Cells

[10] Islets isolated from pancreata human subjects will be cultured and assessed. Donor humans varied in age, were of either gender, and varieties of other clinical factors.

### Cell Growth and Propagation

[11] Islets will be isolated and cultured in either SCT's medium formulation or in standard culture medium (DMEM−low glucose formulation+20% (v/v) Human Serum) as previously described. Because islet cells require more serum in their culture medium, SCT's medium for culture of primary islets was formulated with a serum concentration of 5% (v/v) rather than 1% (v/v) required for A/MSCs.

[12] P0 islet cells will be harvested from such cultures when ~90% confluent using 2× Trypsin EDTA, and assessed for yields, growth rates, and doublings, and then passaged to P1. P1 cultures will be likewise cultured, harvested, and assessed, and performance differences between the SCT255 and standard media will be determined.

[13] SCT's medium is anticipated to consistently support significantly superior and more rapid outgrowth of primary islets, yielding a mean of ~20,000 more P0 islet cells (primary culture) per cm2 in a mean of ~2 fewer days than standard culture medium. Similarly, doubling times are expected to be a mean of 2 hours faster when P0 cells were passaged and cultured to P1.

### Gene Expression

For P0 and P1 islet cells cultured in either SCT's medium formulation or standard culture medium, endocrine gene expression will be assessed by rtPCR. Results for SCT's medium will be normalized to those of the same passage of cells cultured in standard culture medium using the delta-delta ct method, where log 10[RQ]≥±2 is considered statistically significant.

### Example 3 Feline Specific Growth Media

The serum-supplement is feline-derived so that cells that are introduced into the felines are not carriers of xenogeneic proteins that might cause an antigenic response in the recipient. A xeno-free, reduced serum culture medium (SCT255f) variant is in development for the culture of feline cells. In order to prevent xenogeneic immune responses, this medium contains only feline-derived proteins, and feline serum, but no other animal proteins. Two cell types; Adipose-derived or bone marrow-derived Mesenchymal Stromal Cells (A/MSCs), and pancreatic Islet Cells (ICs), are undergoing testing using this medium vs standard, commercially available culture media.

The reduced serum medium for feline M/ASCs and islet cells will be compared head-to-head with standard culture media, either DMEM/F-12+10% (v/v) feline serum, or low glucose DMEM+10% (v/v) feline serum. Cells will be assessed for morphology, growth rates, and gene expression profiles of various beneficial cytokines.

Several variations of the reduced serum medium will be produced and tested. Table 9 depicts the feline specific serum equivalent to the most effective canine serum.

TABLE 10

| Type | Component | Final | |
|---|---|---|---|
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (feline or | 0.17 | uM |
| | feline equivalent) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF feline | 2 | ng/ml |
| | PDGF-BB feline | 2 | ng/ml |
| | EGF-feline | 2 | ng/ml |
| Protein/osmotic | Feline Serum or platelet lysate | 1% | v/v |

TABLE 11

| Type | Component | Final | |
|---|---|---|---|
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (feline or | 0.17 | uM |
| | feline equivalent) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF feline | 10 | ng/ml |
| | PDGF-BB feline | 5 | ng/ml |
| | EGF-feline | 10 | ng/ml |
| Protein/osmotic | Feline Serum or platelet lysate | 1% | v/v |

### Cells Assessed

### Feline M/ASCs

[14] Feline MSCs will be isolated from sub-cutaneous fat (aka ASCs) or bone marrow and examined. Donor felines will vary in age, gender, and other clinical factors.

### Cell Growth

[15] As feline cells share many characteristics with canine cells it is anticipated that the same feline M/ASCs cultured in SCT255f medium, will undergo statistically significantly higher, doublings in the same time frame compared to doublings in standard medium.

### Propagation

[16] feline MSCs will be cultured for 4 to 6 passages in either SCT formulation or standard culture medium. It is anticipated that those cultured in SCT's medium will exhibit faster doubling times and consequently increased number of PDLs observed and will be maintained through 16 doublings.

### Identity

[17] Feline MSCs are in part identified by their expression of cell surface markers. Feline MSCs cultured in SCT's medium are expected to display positive expression certain cell markers and negative expression of other cell markers.

[18] Passaging of cells to >20 PDLS is not expected to affect expression of these markers.

### Gene Expression

[19] Gene expression of beneficial, paracrine cytokines of MSCs cultured in SCT's medium vs. those of the same cells cultured in standard, 10% (v/v) serum containing medium are anticipated to be similar. Expression levels of IFG-1 and IL-6, both involved in immune-modulation by MSCs, is expected to trend higher in cells cultured in SCT's medium than in normal medium, but do not reach statistically significant levels (log 10[RQ]≥±2). IGF-1 expression by MSCs has pro-regenerative and immune modulatory actions, and IL-6 is also immune modulatory.

### Immune Modulation

[20] Cells cultured in SCT's medium are anticipated to respond to overnight INF-γ exposure by inducing expression of IDO-1 and other genes involved in immune modulation. Culture in SCT255 results in the consistent and highly significant up-regulation of the powerful anti-inflammatory cytokine CXCL10.

[21] Secretion of PD-L1 and PD-L2 by human MSCs has been shown to directly affect T cell behavior, induce immune tolerance, and mediate antifibrotic effects. upon exposure to INF-γ, responses in feline cells are anticipated to be similar. PD-L1 is expected to be more highly expressed in cells cultured in SCT 255 than in standard medium. PD-L1 expression by cancer cells is known to induce tolerance and allow such cells to evade destruction by the immune system. Interruption of the PD-1/PD-L1 pathway is currently used as an anti-cancer therapeutic in humans.

### Feline Islet Cells

[22] Islets isolated from pancreata feline subjects will be cultured and assessed. Donor felines will vary in age, gender, and varieties of other clinical factors.

### Cell Growth and Propagation

[23] Islets will be isolated and cultured in either SCT's medium formulation or in standard culture medium (DMEM−low glucose formulation+20% (v/v) Feline Serum) as previously described. Because islet cells require more serum in their culture medium, SCT's medium for culture of primary islets was formulated with a serum concentration of 5% (v/v) rather than 1% (v/v) required for A/MSCs.

[24] P0 islet cells will be harvested from such cultures when ~90% confluent using 2× Trypsin EDTA, and assessed for yields, growth rates, and doublings, and then passaged to P1. P1 cultures will be likewise cultured, harvested, and assessed, and performance differences between the SCT255f and standard media will be determined.

[25] SCT's medium is anticipated to consistently support significantly superior and more rapid outgrowth of primary islets, yielding a mean of ~20,000 more P0 islet cells (primary culture) per cm2 in a mean of ~2 fewer days than standard culture medium. Similarly, doubling times are expected to be a mean of 2 hours faster when P0 cells were passaged and cultured to P1.

### Gene Expression

For P0 and P1 islet cells cultured in either SCT's medium formulation or standard culture medium, endocrine gene expression will be assessed by rtPCR. Results for SCT's medium will be normalized to those of the same passage of cells cultured in standard culture medium using the delta-delta ct method, where log 10[RQ]≥±2 is considered statistically significant.

### Example 4 Equine Specific Growth Media

The serum-supplement is feline-derived so that cells that are introduced into the equines are not carriers of xenogeneic proteins that might cause an antigenic response in the recipient. A xeno-free, reduced serum culture medium (SCT255e) variant is in development for the culture of equine cells. In order to prevent xenogeneic immune responses, this medium contains only equine-derived proteins, and equine serum, but no other animal proteins. Two cell types; Adipose-derived or bone marrow-derived Mesenchymal Stromal Cells (A/MSCs), and pancreatic Islet Cells (ICs), are undergoing testing using this medium vs standard, commercially available culture media.

The reduced serum medium for feline M/ASCs and islet cells will be compared head-to-head with standard culture media, either DMEM/F-12+10% (v/v) equine serum, or low glucose DMEM+10% (v/v) equine serum. Cells will be assessed for morphology, growth rates, and gene expression profiles of various beneficial cytokines.

Several variations of the reduced serum medium will be produced and tested. Table 11 depicts the feline specific serum equivalent to the most effective canine serum.

TABLE 12

| Type | Component | Final | |
|---|---|---|---|
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (equine or | 0.17 | uM |
| | equine equivalent) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF equine | 2 | ng/ml |
| | PDGF-BB equine | 5 | ng/ml |
| | EGF-equine | 5 | ng/ml |
| Protein/osmotic | Equine Serum or platelet lysate | 1% | v/v |

Alternative formulations of a reduced serum medium were also tested, and these compositions are also included as Table 13.

TABLE 13

| Type | Component | Final | |
|---|---|---|---|
| | Water | | |
| Powder Medium | DMEM/F12 | | |
| Buffering agents | Sodium Bicarbonate | 28.6 | mM |
| | HEPES (1M) | 5 | mM |
| Amino Acid | L-glutamine | 3.25 | mM |
| | L-alanine | 0.8 | mM |
| Lipids | Lipid Concentrate | 0.10% | v/v |
| Hormones | Insulin (equine or | 0.17 | uM |
| | equine equivalent) | | |
| | Hydrocortisone | 100 | nM |
| | (plant, wild yam) | | |
| | Progesterone (synthetic) | 17 | nM |
| Polyamides | Putrescine (microbial) | 56.05031 | μm |
| Vitamin/antioxidant | Asc-2-P (Vit C) | 200 | μm |
| Growth Factors | bFGF equine | 10 | ng/ml |
| | PDGF-BB equine | 5 | ng/ml |
| | EGF-equine | 10 | ng/ml |
| Protein/osmotic | Equine Serum or platelet lysate | 1% | v/v |

### Cells Assessed

### Equine M/ASCs

[26] Equine MSCs will be isolated from sub-cutaneous fat (aka ASCs) or bone marrow and examined. Donor Equines will vary in age, gender, and other clinical factors.

### Cell Growth

[27] As equine cells share many characteristics with canine cells it is anticipated that the same equine M/ASCs cultured in SCT255e medium, will undergo statistically significantly higher, doublings in the same time frame compared to doublings in standard medium

### Propagation

[28] Equine MSCs will be cultured for 4 to 6 passages in either SCT formulation or standard culture medium. It is anticipated that those cultured in SCT's medium will exhibit faster doubling times and consequently increased number of PDLs observed and will be maintained through 16 doublings.

### Identity

[29] Equine MSCs are in part identified by their expression of cell surface markers. Feline MSCs cultured in SCT's medium are expected to display positive expression certain cell markers and negative expression of other cell markers.

[30] Passaging of cells to >20 PDLS is not expected to affect expression of these markers.

### Gene Expression

[31] Gene expression of beneficial, paracrine cytokines of MSCs cultured in SCT's medium vs. those of the same cells cultured in standard, 10% (v/v) serum containing medium are anticipated to be similar. Expression levels of IFG-1 and IL-6, both involved in immune-modulation by MSCs, is expected to trend higher in cells cultured in SCT's medium than in normal medium, but do not reach statistically significant levels (log 10[RQ]≥±2). IGF-1 expression by MSCs has pro-regenerative and immune modulatory actions, and IL-6 is also immune modulatory.

### Immune Modulation

[32] Cells cultured in SCT's medium are anticipated to respond to overnight INF-γ exposure by inducing expression of IDO-1 and other genes involved in immune modulation. Culture in SCT255 results in the consistent and highly significant up-regulation of the powerful anti-inflammatory cytokine CXCL10.

[33] Secretion of PD-L1 and PD-L2 by human MSCs has been shown to directly affect T cell behavior, induce immune tolerance, and mediate antifibrotic effects. upon exposure to INF-γ, responses in equine cells are anticipated to be similar. PD-L1 is expected to be more highly expressed in cells cultured in SCT 255 than in standard medium. PD-L1 expression by cancer cells is known to induce tolerance and allow such cells to evade destruction by the immune system. Interruption of the PD-1/PD-L1 pathway is currently used as an anti-cancer therapeutic in humans.

### Equine Islet Cells

[34] Islets isolated from pancreata equine subjects will be cultured and assessed. Donor equines will vary in age, gender, and varieties of other clinical factors.

### Cell Growth and Propagation

[35] Islets will be isolated and cultured in either SCT's medium formulation or in standard culture medium (DMEM−low glucose formulation+20% (v/v) Equine Serum) as previously described. Because islet cells require more serum in their culture medium, SCT's medium for culture of primary islets was formulated with a serum concentration of 5% (v/v) rather than 1% (v/v) required for A/MSCs.

[36] P0 islet cells will be harvested from such cultures when ~90% confluent using 2× Trypsin EDTA, and assessed for yields, growth rates, and doublings, and then passaged to P1. P1 cultures will be likewise cultured, harvested, and assessed, and performance differences between the SCT255e and standard media will be determined.

[37] SCT's medium is anticipated to consistently support significantly superior and more rapid outgrowth of primary islets, yielding a mean of ~20,000 more P0 islet cells (primary culture) per cm2 in a mean of ~2 fewer days than standard culture medium. Similarly, doubling times are expected to be a mean of 2 hours faster when P0 cells were passaged and cultured to P1.

### Gene Expression

For P0 and P1 islet cells cultured in either SCT's medium formulation or standard culture medium, endocrine gene expression will be assessed by rtPCR. Results for SCT's medium will be normalized to those of the same passage of cells cultured in standard culture medium using the delta-delta ct method, where log 10[RQ]≥±2 is considered statistically significant.

The invention has been described with reference to various specific and preferred embodiments and techniques. Nevertheless, it is understood that many variations and modifications may be made while remaining within the spirit and scope of the invention.

### REFERENCES (INCORPORATED BY REFERENCE HEREIN)

There were other references, too from the submitted document which may be of use here.
1. Silva D N, Souza B S F, Azevedo C M, Vasconcelos J F, de Jesus P G, Feitoza M S, et al. IGF-1-Overexpressing Mesenchymal Stem/Stromal Cells Promote Immunomodulatory and Proregenerative Effects in Chronic Experimental Chagas Disease. Liu J, editor. Stem Cells Int [Internet]. 2018; 2018:9108681. Available from: https://doi.org/10.1155/2018/9108681
2. Chen L, Tredget E E, Wu P Y G, Wu Y. Paracrine factors of mesenchymal stem cells recruit macrophages and endothelial lineage cells and enhance wound healing. PLOS One [Internet]. 2008 Apr. 2; 3 (4): e1886. Available from: http://www.ncbi.nlm.nih.gov/pubmed/18382669
3. Spees J L, Gregory C A, Singh H, Tucker H A, Peister A, Lynch P J, et al. Internalized Antigens Must Be Removed to Prepare Hypoimmunogenic Mesenchymal Stem Cells for Cell and Gene Therapy. 2004; 9 (5): 747-56.

### REFERENCES with PROTOCOLS FOR ISOLATION AND EXPANSION OF MSC/ASC (INCORPORATED BY REFERENCE HEREIN)

5. Toegel F, Hu Z, Weiss K, Isaac J, Lange C, Westenfelder C: Administered mesenchymal stem cells protect against ischemic acute renal failure through differentiation-independent mechanisms. Am J Physiol Renal Physiol. 2005 July; 289(1):F31-42. Epub 2005 Feb. 15
6. Tögel F, Weiss K, Yang Y, Hu Z, Zhang P, Westenfelder C: Vasculotropic, paracrine actions of infused mesenchymal stem cells are important to the recovery from acute kidney injury. Am J Physiol Renal Physiol. 2007 May; 292(5):F1626-35. Epub 2007 Jan. 9.

### REFERENCES with PROTOCOLS FOR ISOLATION OF MSC/ASC-derived EXOSOMES (INCORPORATED BY REFERENCE HEREIN)

7. Lötvall J, Hill A F, Hochberg F et al.: Minimal experimental requirements for definition of extracellular vesicles and their functions: a position statement from the International Society for Extracellular Vesicles. Journal of Extracellular Vesicles 2014, 3:26913-http://dx.doi.org/10.3402/jev.v3.26913
8. Bang C, Thum T: Exosomes: New players in cell=cell communication. Int J Biochem Cell Biol 2012; 44:2060-2064.
9. Kholina S, Ranghino A, Garnieri P et al.: Extracellular vesicles as new players in angiogenesis. Vasc Pharmacol 2016; S1537-1891:30105-3-101.

1. A species-specific cell culture medium comprising:
basal medium, sodium bicarbonate, HEPES, L-glutamine, L-alanine, insulin, hydrocortisone, progesterone, Asc-2-p, holo-transferrin; bFGF of the species, PDGF-BB of the species, and EGF of the species; and serum of the species.

2. The species-specific cell culture medium of claim 1, wherein the species-specific cell culture medium comprises between about 1 percent and about 20 percent by volume of the serum of the species.

3. (canceled)

4. (canceled)

5. The species-specific cell culture medium of claim 1, wherein the insulin is insulin of the species.

6. The species-specific cell culture medium of claim 1, wherein:
the bFGF is canine bFGF, the PDGF-BB is canine PDGF-BB, the EGF is canine EGF; and the serum is canine serum.

7. (canceled)

8. (canceled)

9. (canceled)

10. A composition comprising:
the species-specific cell culture medium of claim 1; and
cells of the species.

11. The composition of claim 10, wherein the species is Homo sapiens or canine.

12. The composition of claim 10, wherein the cells of the species are mesenchymal stem cells.

13. The composition of claim 12, wherein the mesenchymal stem cells comprise adipose stromal cells.

14. The composition of claim 12, wherein the composition further comprises islet cells of the species.

15. the composition of claim 10, wherein the cells are islet cells of the species.

16. (canceled)

17. (canceled)

18. (canceled)

19. (canceled)

20. (canceled)

21. A method for culturing and propagating cells of a species, the method comprising:
culturing cells of the species in the species-specific cell culture medium of claim 1 under conditions for propagating the cells, wherein the culture medium is specific for the species.

22. The method of claim 21, wherein the cells comprise mesenchymal stromal cells, adipose stromal cells, and/or islet cells.

23. The method of claim 21, wherein the species is canine or human.

24. (canceled)

25. (canceled)

26. (canceled)

27. (canceled)

28. (canceled)

29. (canceled)

30. The method according to claim 21, the method further comprising:
collecting exosomes released by the cells in culture.

31. The method of claim 30, wherein the cells are mesenchymal cells.

32. The method of claim 30, wherein the cells are islet cells.

33. The method according to claim 30, further comprising:
treating a subject of the species with the collected exosomes.

34. The method according to claim 33, wherein the mesenchymal and/or islet stem cells are allogeneic to the subject.
